SEQUENCE LISTING (44 sequences)

SEQ ID NO: 1 - DNA, 24 bp, Artificial Sequence
  Primer Forward Sequence
cctcttgaccggcgtgtaacggcg

SEQ ID NO: 2 - DNA, 23 bp, Artificial Sequence
  Primer Reverse Sequence
ctccacatcactgtcttgcttcc

SEQ ID NO: 3 - DNA, 23 bp, Artificial Sequence
  Primer Forward Sequence
gcattacagcggaagttttcgga

SEQ ID NO: 4 - DNA, 22 bp, Artificial Sequence
  Primer Reverse Sequence
cacaccgtatcatgcgatactg

SEQ ID NO: 5 - DNA, 23 bp, Artificial Sequence
  Primer Forward Sequence
gatttgaatgaagttttcggatg

SEQ ID NO: 6 - DNA, 22 bp, Artificial Sequence
  Primer Reverse Sequence
cctgcaccatgcggcgctgtgg

SEQ ID NO: 7 - DNA, 23 bp, Artificial Sequence
  Primer Forward Sequence
gaacgaagcaatttaacggaagt

SEQ ID NO: 8 - DNA, 22 bp, Artificial Sequence
  Primer Reverse Sequence
cacactgtatcatgcgatactg

SEQ ID NO: 9 - DNA, 23 bp, Artificial Sequence
  Primer Forward Sequence
ggtttcgatgaagttttcggatg

SEQ ID NO: 10 - DNA, 22 bp, Artificial Sequence
  Primer Reverse Sequence
caccagaccatgcggccctgtg

SEQ ID NO: 11 - DNA, 23 bp, Artificial Sequence
  Primer Forward Sequence
gcacttgagcggatttcttcgga

SEQ ID NO: 12 - DNA, 22 bp, Artificial Sequence
  Primer Reverse Sequence
cacaccagaccatgcggtcctg

SEQ ID NO: 13 - DNA, 23 bp, Artificial Sequence
  Primer Forward Sequence
gcggatttcttcggattgaagca

SEQ ID NO: 14 - DNA, 22 bp, Artificial Sequence
  Primer Reverse Sequence
cacacggtaccatgcggtactg

SEQ ID NO: 15 - DNA, 23 bp, Artificial Sequence
  Primer Forward Sequence
gcatttaggattgaagttttcgg

SEQ ID NO: 16 - DNA, 22 bp, Artificial Sequence
  Primer Reverse Sequence
cacactgaatcatgcgattctg

SEQ ID NO: 17 - DNA, 23 bp, Artificial Sequence
  Primer Forward Sequence
gatagttagaatgagagcttcgg

SEQ ID NO: 18 - DNA, 22 bp, Artificial Sequence
  Primer Reverse Sequence
cttcctcagaagatgccttccg

SEQ ID NO: 19 - DNA, 22 bp, Artificial Sequence
  Primer Forward Sequence
gacgcgagcacttgtgctcgag

SEQ ID NO: 20 - DNA, 22 bp, Artificial Sequence
  Primer Reverse Sequence
cggtcaccatgcagtgtccgta

SEQ ID NO: 21 - DNA, 23 bp, Artificial Sequence
  Primer Forward Sequence
gaacgcactgattttatcagtga

SEQ ID NO: 22 - DNA, 22 bp, Artificial Sequence
  Primer Reverse Sequence
gaagcggtcatgcgaccccttc

SEQ ID NO: 23 - DNA, 23 bp, Artificial Sequence
  Primer Forward Sequence
ggtagcttgctatcggagcttag

SEQ ID NO: 24 - DNA, 22 bp, Artificial Sequence
  Primer Reverse Sequence
cagagaagagatgcctcctctc

SEQ ID NO: 25 - DNA, 23 bp, Artificial Sequence
  Primer Forward Sequence
gtttcgaggaagcttgcttccaa

SEQ ID NO: 26 - DNA, 22 bp, Artificial Sequence
  Primer Reverse Sequence
ctgagcatgcgctctgtatacc

SEQ ID NO: 27 - DNA, 23 bp, Artificial Sequence
  Primer Forward Sequence
gaggggagcttgctccccagagc

SEQ ID NO: 28 - DNA, 22 bp, Artificial Sequence
  Primer Reverse Sequence
gataccagaatcatgcggtccc

SEQ ID NO: 29 - DNA, 23 bp, Artificial Sequence
  Primer Forward Sequence
gttaagagagcttgctcttttaa

SEQ ID NO: 30 - DNA, 22 bp, Artificial Sequence
  Primer Reverse Sequence
ggtcgctgtaccatgcgatact

SEQ ID NO: 31 - DNA, 23 bp, Artificial Sequence
  Primer Forward Sequence
ggaaatctcttcggagatggaat

SEQ ID NO: 32 - DNA, 22 bp, Artificial Sequence
  Primer Reverse Sequence
gacgttcaagagatgcctccca

SEQ ID NO: 33 - DNA, 23 bp, Artificial Sequence
  Primer Forward Sequence
gctcatgacggaggattcgtcca

SEQ ID NO: 34 - DNA, 22 bp, Artificial Sequence
  Primer Reverse Sequence
ggcagtcagagccatgcgaccc

SEQ ID NO: 35 - DNA, 22 bp, Artificial Sequence
  Primer Forward Sequence
ggcagtcagagccatgcgaccc

SEQ ID NO: 36 - DNA, 22 bp, Artificial Sequence
  Primer Reverse Sequence
catgcggacatgtgaactcatg

SEQ ID NO: 37 - DNA, 22 bp, Artificial Sequence
  Primer Forward Sequence
ccctggcagcttgctgccgggg

SEQ ID NO: 38 - DNA, 22 bp, Artificial Sequence
  Primer Reverse Sequence
cactcgcatgcgctcatgtgga

SEQ ID NO: 39 - DNA, 23 bp, Artificial Sequence
  Primer Forward Sequence
gatccatcaagcttgcttggtgg

SEQ ID NO: 40 - DNA, 22 bp, Artificial Sequence
  Primer Reverse Sequence
cacaccatgcagtgtgatggag

SEQ ID NO: 41 - DNA, 21 bp, Artificial Sequence
  Primer Forward Sequence
actcctacgggaggcagcagt

SEQ ID NO: 42 - DNA, 18 bp, Artificial Sequence
  Primer Reverse Sequence
attaccgcggctgctggc

SEQ ID NO: 43 - DNA, 24 bp, Artificial Sequence
  Primer Forward Sequence
gacgctgaggcatgatgagagcat

SEQ ID NO: 44 - DNA, 21 bp, Artificial Sequence
  Primer Reverse Sequence
gacggcacggattgttattca

(19) United States
(12) Patent Application Publication
(10) Pub. No.: US 2019/0078143 A1
(43) Pub. Date: Mar. 14, 2019

(54) METHOD FOR TREATING NEURODEVELOPMENTAL DISORDERS

(71) Applicants: New York University, New York, NY (US); University of Massachusetts, Worchester, MA (US); Massachusetts Institute of Technology, Cambridge, MA (US)

(72) Inventors: Dan R. Littman, New York, NY (US); Jun R. Huh, Worchester, MA (US); Gloria B. Choi, Cambridge, MA (US); Sangdoo Kim, Worchester, MA (US)

(21) Appl. No.: 16/128,078

(22) Filed: Sep. 11, 2018

Related U.S. Application Data
(60) Provisional application No. 62/556,854, filed on Sep. 11, 2017.

(51) Int. Cl. C12Q 1/689; A61K 38/14; A61K 31/197; A61K 31/42; A61K 38/17; A61P 25/00

(52) U.S. Cl. CPC C12Q 1/689; A61K 38/14; A61P 25/00; A61K 31/42; A61K 38/1709; A61K 31/197

(57) ABSTRACT

Provided is a method for identifying and reducing the risk of a female subject producing an offspring having a neurodevelopmental disorder. The method comprises detecting in the subject the presence of gut bacteria that promote Th17 cell biogenesis. If such bacteria are detected, the individual can be administered a therapy that inhibits the growth or Th17 cell biogenesis promoting activity of the bacteria, and/or reduces the activity of the Th17 cells in the gut.

### CROSS REFERENCE TO RELATED APPLICATION

This application claims priority to U.S. Provisional application No. 62/556,854, filed on Sep. 11, 2017, the disclosure of which is incorporated herein by reference.

### STATEMENT REGARDING FEDERALLY SPONSORED RESEARCH

This invention was made with government support under contract numbers R01DK106351 and R01DK110559 awarded by the National Institutes of Health. The government has certain rights in the invention.

### BACKGROUND OF THE DISCLOSURE

Maternal immune activation (MIA) contributes to behavioral abnormalities associated with neurodevelopmental disorders in both primate and rodent offspring (Machado et al., Biological psychiatry, 77:823-832, (2015); Bauman et al., Biological psychiatry, 75:332-341, (2014); Smith et al., The Journal of neuroscience, 27:10695-10702 (2007); Malkova et al., Brain Behav Immun, 26:607-616, (2012)). In humans, epidemiological studies suggest that exposure of fetuses to maternal inflammation increases the likelihood of developing Autism Spectrum Disorder (ASD) (Lee et al. Brain, behavior, and immunity 44; 100-105, (2015); Brown et al. Molecular psychiatry, 19:259-264, (2014); Atladottir et al. J Autism Dev Disord, 40:1423-1430, (2010). Recently it has been demonstrated that interleukin-17a (IL-17a) produced by Th17 cells, CD4⁺ T helper effector cells involved in multiple inflammatory conditions, is required in pregnant mice to induce behavioral as well as cortical abnormalities in the offspring exposed to MIA (Choi et al. Science 351, 933-939, (2016)). However, it is unclear if other maternal factors are required to promote MIA-associated phenotypes. Moreover, underlying mechanisms by which MIA leads to T cell activation with increased IL-17a in the maternal circulation are not well understood.

### SUMMARY OF THE DISCLOSURE

In the present disclosure, the ability of commensal microbiota on the mother's likelihood of producing offspring with MIA-associated phenotypes was determined. We show that MIA phenotypes in offspring require maternal intestinal bacteria that promote Th17 cell biogenesis (growth and/or differentiation). Pregnant mice that had been colonized with the mouse commensal segmented filamentous bacteria (SFB) or human commensal bacteria that induce intestinal Th17 cells were more likely to produce offspring with MIA-associated abnormalities. We also show that small intestine dendritic cells (DCs) from pregnant, but not from non-pregnant, females upon exposure to MIA secrete IL-113/IL-23/IL-6 and stimulate T cells to produce IL-17a. Our data indicate that defined gut commensal bacteria with a propensity to induce Th17 cells increase the risk for neurodevelopmental disorders in offspring of pregnant mothers undergoing immune system activation due to infections or auto-inflammatory syndromes.

The present disclosure provides methods for identifying the presence of Th17 cell biogenesis (growth and/or differentiation) promoting bacteria in the gut of a female subject. The presence of such bacteria is an indication that the female subject is at risk of producing an offspring with a neurodevelopmental disorder, such as, but not limited to autism. The present methods can also comprise steps of identifying suitable agents effective to combat the presence or activity of such bacteria. The present methods can further comprise the step of administering suitable agents effective to combat the presence or activity of such bacteria to the female subject.

The present method of screening for the presence of Th17 cell biogenesis promoting bacteria in the gut of a female subject can be carried out as a monitoring test prior to pregnancy or during pregnancy, and can be carried out at any desired frequency.

### BRIEF DESCRIPTION OF THE FIGURES

FIG. 1. Maternal bacteria promote abnormal behaviors associated with neurodevelopmental disorders in MIA offspring. a, Ultrasonic vocalization (USV) index (n=28/34 (vehicle;PBS/poly(I:C)); n=26/30 (vancomycin;PBS/poly(I:C)); 5-6 independent experiments). b-d, Marble-burying index (b) time spent in the center of an open field (c), % interaction (d) in the sociability test of adult offspring described in (a) (n=13/15 (vehicle;PBS/poly(I:C)); n=12/16 (vancomycin;PBS/poly(I:C)); 3-4 independent experiments). e, Representative images of adult offspring brains from PBS-/poly(I:C)-injected mothers treated with vehicle/vancomycin. Arrows indicate cortical patch. Scale bar, 100 μm (n=3/4 (PBS;vehicle/vancomycin); n=5/4 (poly(I:C);vehicle/vancomycin); 2 independent experiments). f, Maternal plasma concentrations of IL-17a 48 hrs after PBS/poly(I:C) administration into dams at E12.5 (n=6/group; 3 independent experiments). *p<0.05, **p<0.01, ***p<0.001, ****p<0.0001 as calculated by one-way (a-c) and two-way (d) ANOVA with Tukey post-hoc tests and Student's t-test (f). N.S., not significant. Graphs indicate mean+/−s.e.m.

FIG. 2. SFB in the pregnant mothers promotes abnormal behaviors in MIA offspring. a, USV index (n=59/125 (Tac;PBS/poly(I:C)); n=51/50 (Jax;PBS/poly(I:C)); n=55/81 (Co-housed Jax;PBS/poly(I:C)); n=55/89 (SFB-gavaged Jax;PBS/poly(I:C)); 9-11 independent experiments). b-d, Marble burying index (b), time spent in the center of an open field (c), and % interaction (d) in the sociability assay of adult offspring described in (a) (n=32/50 (Tac;PBS/poly(I:C)); n=29/27 (Jax;PBS/poly(I:C)); n=29/29 (Co-housed Jax;PBS/poly(I:C)); n=33/30 (SFB-gavaged Jax;PBS/poly(I:C)); 7-8 independent experiments). e, Representative images of adult offspring brains from PBS-/poly(I:C)-injected mothers. Arrows indicate cortical patches. Scale bar, 100 μm (n=3/3 (PBS;Tac/Jax); n=3/3 (PBS;co-housed Jax/SFB-gavaged Jax); n=4/3 (poly(I:C);Tac/Jax); n=3/3 (poly(I:C);co-housed Jax/SFB-gavaged Jax)). f, Maternal plasma concentrations of IL-17a 48 hrs after administration of PBS/poly(I:C) into dams at E12.5 (n=6/group; 2 independent experiments). ***p<0.001, ****p<0.0001 as calculated by one-way (a-c) and two-way (d) ANOVA with Tukey post-hoc tests and Student's t-test (f). N.S., not significant. Graphs indicate mean+/−s.e.m.

FIG. 3. SFB-specific T cells are the major IL-17a producer in pregnant mothers treated with poly(I:C). a-e,g, Supernatant concentrations of IL-17a from ex vivo cultured mononuclear cells of ilea in PBS/poly(I:C)-treated dams (a) (n=4-5/group), from co-culture of CD4⁺ and CD11c⁺ of ilea in PBS/poly(I:C)-treated Tac/Jax mice (b) (n=4/group), from co-cultures of CD4⁺ and CD11c⁺ of ilea in poly(I:C)-treated WT/TLR3 KO mice (c) (n=4-6/group), from co-cultures of GFP⁺CD4⁺/GFP⁻CD4⁺ and CD11c⁺ from ilea of poly(I:C)-treated il17a^{gfp} mice (d) (n=8/group), from sorted GFP⁺/GFP⁻CD4⁺ cells (e) (n=6/group), or from co-cultures of CD4⁺ and CD11c⁺ (g) (n=4/group). CD4^{Sp} indicates spleen-derived CD4⁺ T cells. All cultures were isolated at E14.5 and stimulated with poly(I:C) for 18 hrs (a-c,g) or for 48 hrs (d-e). f, Maternal plasma concentrations of IL-17a 48 hrs after administration of PBS/poly(I:C) into non-pregnant females or dams at E12.5 (n=4/5 (non-pregnant females;PBS/poly(I:C)); n=4/5 (pregnant females;PBS/poly(I:C))). All data pooled from 2 independent experiments. **p<0.01, ****p<0.0001 as calculated by one-way (a-f) ANOVA with Tukey post-hoc tests and Student's t-test (g); N.D., not determined. N.S., not significant. Graphs indicate mean+/−s.e.m. In FIG. 3d, the bars from left to right for each set (GFP(−) CD4+ cells, and GFP(+) CD4+ cells) are: Vehicle; Isotype Ab; Isotype Ab+Poly(I:C); and Anti-IL-6, -IL-1b, -IL-23 Ab+Poly(I:C). In FIG. 3e, the bars from left to right for each set (GFP(−) CD4+ cells, and GFP(+) CD4+ cells) are: Vehicle; Poly(I:C); and IL-6, IL-1b, IL-23.

FIG. 4. Human commensal bacteria inducing gut Th17 cells promote abnormal behavioral phenotypes in MIA offspring. a, USV index (n=38/32/27 for vehicle-gavaged only/human bacteria-gavaged+isotype control antibody/human bacteria-gavaged+anti-IL-17a antibody; 6 independent experiments). b-d, Marble burying index (b), time spent in the center of an open-field (c), and % interaction (d) (n=23/22/13 for vehicle-gavaged only/human bacteria-gavaged+isotype control antibody/human bacteria-gavaged+anti-IL-17a antibody; 4 independent experiments). e, Representative SATB2 staining in the cortex of the offspring derived from vehicle/human bacteria-gavaged Jax dams. Arrows indicate cortical patches. Scale bar, 100 μm, f. Maternal plasma concentrations of IL-17a at E14.5 (n=7-14/group; 2 independent experiments). *p<0.05, ****p<0.0001 as calculated by one-way (a-c, f) or two-way (d) ANOVA with Tukey post-hoc tests. Graphs indicate mean+/−s.e.m.

FIG. 5. Maternal vancomycin-treatment prevented induction of behavioral abnormalities in MIA offspring. a, USV index (n=27/29 (PBS;male/female); n=28/21 (Poly(I:C);male/female); 6 independent experiments). b-c, Total investigation time (b) and total distance traveled (c) during the sociability test (n=13/15 (vehicle;PBS/poly(I:C)); n=12/16 (vancomycin;PBS/poly(I:C)); 3-4 independent experiments). d, Schematic of the experimental design. e-f, Quantification of SATB2⁺ cells (e) in the cortex divided into ten equal bins representing different depths of the cortex or of the cortical patch size (f) in the primary somatosensory cortex (S1) (n=3/4 (PBS;vehicle/vancomycin); n=3/4 (poly(I:C);vehicle/vancomycin); 2 independent experiments). g, Flow cytometry of CD4⁺ T cells (gated on TCR-β⁺CD4⁺) stained intracellularly for IL-17a and RORγt. Mononuclear cells were collected at E14.5 from the ilea of poly(I:C)-treated mice with/without vancomycin treatment; Representative FACS plot from 3 independent experiments. h, qPCR analysis measuring relative SFB levels in B6 mice before/after vancomycin treatments (n=4-5/group). i, Representative SEM images of epithelial surfaces in the ilea of the vehicle-/vancomycin-treated mice from 2 independent experiments. Scale bars, 30 μm. *p<0.05, **p<0.01, ***p<0.001, ****p<0.0001 as calculated by two-way (a,e) and one-way (b,c,f) ANOVA with Tukey post-hoc tests. N.D., not determined; N.S., not significant. Graphs indicate mean+/−s.e.m.

FIG. 6. MIA in SFB-absent Jax mothers does not induce changes in the total activity of the adult offspring, properties of the litter and maternal cytokine production. a-b, Total investigation time (a) and total distance traveled (b) during the sociability test. c, Litter size upon weaning (n=59/125 (Tac;PBS/poly(I:C)); n=51/50 (Jax;PBS/poly(I:C)); n=55/81 (Co-housed Jax;PBS/poly(I:C)); n=55/89 (SFB-gavaged Jax;PBS/poly(I:C)). d, Weight of male offspring from the groups described in (c) (n=32/50 (Tac;PBS/poly(I:C)); n=29/27 (Jax;PBS/poly(I:C)); n=29/29 (Co-housed Jax;PBS/poly(I:C)); n=33/30 (SFB-gavaged Jax;PBS/poly(I:C)). Data in a, b, and d are from 7-8 independent experiments. e-f, Quantification of SATB2⁺ cells (e) in the cortex divided into ten equal bins representing different depth and of patch size (f) in the S1 (n=4 (Tac;PBS); n=3/3/4/3 (Tac/Jax/Co-housed Jax/SFB-gavaged Jax;poly(I:C)). g, Maternal plasma concentrations of TNF-α and IFN-β at 3 hrs after PBS/poly(I:C) injection into Tac/Jax dams at E12.5; n=4/group. *p<0.05, **p<0.01, ***p<0.001 as calculated by two-way (e) and one-way ANOVA (a-d,g,f) with Tukey post-hoc tests and Student's t-test (g). N.D., not determined. Graphs indicate mean+/−s.e.m.

FIG. 7. SFB colonization leads to increased levels of gut Th17 cells in Jax pregnant mice. a, Schematic of the experimental design. b. Flow cytometry of CD4⁺ T cells (gated on TCR-β⁺CD4⁺) stained intracellularly for IL-17a and RORγt. Mononuclear cells were collected at E14.5 from the ilea of poly(I:C)-treated Tac/Jax/co-housed Jax/SFB-gavaged Jax mothers. c, Representative SEM images of epithelial surfaces in the ilea of Tac/Jax/co-housed Jax/SFB-gavaged Jax mothers. Scale bars, 30 μm. Data representative of 3 (b) and 2 (c) independent experiments. d, qPCR analysis for SFB levels in the fecal samples of the groups described in (a) (n=4-5/group). ****p<0.0001 as calculated by one-way (d) ANOVA with Tukey post-hoc test. Graphs indicate mean+/−s.e.m.

FIG. 8. Poly(I:C)-induced inflammation during pregnancy, not after giving birth, is critical in inducing MIA-associated behavioral abnormalities in offspring. a, Schematic of the experimental design for cross-fostering experiments. b, USV index (n=21/20 (PBS dams;PBS/poly(I:C) pups); n=22/15 (poly(I:C) dams;PBS/poly(I:C) pups); 2-4 independent experiments). c-g Marble-burying index (c), time spent in the center of an open field (d), and % interaction (e), total investigation time (f), and total distance traveled (g) during the sociability test (n=9/14 (PBS dams;PBS/poly(I:C) pups); n=12/10 (poly(I:C) dams;PBS/poly(I:C) pups); 2 independent experiments). **p<0.01, ****p<0.0001 as calculated by one-way (b-d,f-g) and two-way (e) ANOVA with Tukey post-hoc tests. N.S., not significant. Graphs indicate mean+/−s.e.m.

FIG. 9. Composition of maternal gut microbiota during pregnancy, not after giving birth, is critical in inducing MIA-associated behavioral abnormalities in offspring. a, Schematic of the experimental design for cross-fostering experiments. b, USV index (n=9/36 (Tac pups with Jax dams;PBS/poly(I:C)); n=10/24 (Jax pups with Tac dams;PBS/poly(I:C)); 2-4 independent experiments). c-g, Marble-burying index (c), time spent in the center of an open field (d), and % interaction (e), total investigation time (f), and total distance traveled (g) during the sociability test (n=7/22 (Tac pups with Jax dams;PBS/poly(I:C)); n=7/21 (Jax pups with Tac dams;PBS/poly(I:C)); 2 independent experiments). *p<0.05, **p<0.01, ***p<0.001, ****p<0.0001 as calculated by one-way (b-d,f-g) and two-way (e) ANOVA with Tukey post-hoc tests. Graphs indicate mean+/−s.e.m.

FIG. 10. CD11c⁺ cells stimulate gut-Th17 cells to produce high levels of IL-17a ex vivo. a-f, Flow cytometry of CD4⁺ T cells (gated on TCR-β⁺CD4⁺) stained intracellularly for IL-17a and RORγt. Mononuclear cells were collected at E14.5 from the gut ilea, spleens, and mesenteric lymph nodes (mLN) of PBS-/poly(I:C)-treated mice (n=5/group (a, c, e); n=3/group (b, d, f)). MFI denotes mean fluorescence intensity. g-i, Supernatant concentrations of IL-17a from mononuclear cells of the ilea in poly(I:C)-treated Tac dams (g) (n=3/group), from co-cultures of CD4⁺ and non-CD4⁺ cells of the ilea in PBS-/poly(I:C)-treated Tac dams (h) (n=3/group), or from co-cultures of CD4⁺ and CD103⁻CD11b⁺/CD103⁺CD11b⁺/CD103⁺CD11b⁻ (gated on MHCII⁺CD11c⁺ cells of the ilea in poly(I:C)-treated dams (i) (n=7/group). All cultures were isolated at E14.5 and stimulated ex vivo with poly(I:C) for 18 hrs (g-h) or for 48 hrs (i). Data are pooled from 2 (g-h) or 3 (i) independent experiments. j. USV index (n=16/17 (poly(I:C);WT/TLR3 KO); 2 independent experiments). k, Supernatant concentrations of IL-6, IL-113, and IL-23 from cultures of CD11c⁺ isolated at E14.5 from the ilea of poly(I:C)-treated non-pregnant/pregnant mice (n=5/group; 3 independent experiments). *p<0.05, **p<0.01, ***p<0.001 and ****p<0.0001 as calculated by Student's t-test (a-f,j,k) and one-way ANOVA (g-i) with Tukey post-hoc tests. N.S., not significant. Graphs indicate mean+/−s.e.m.

FIG. 11. SFB-specific 7B8 Tg CD4⁺ T cells produce IL-17a upon transfer to MIA-exposed pregnant mothers. a, Schematic of the experimental design. b-c, Both TCRα KO and IL-17a KO females, with or without adoptive transfers of 7B8 Tg-derived CD4⁺ T cells, were crossed with B6 WT males to produce heterozygous WT offspring. USV index (n=16/30 (TCRa;poly(I:C)/7B8 Tg T cell transfer); n=23/23 (IL-17a KO;poly(I:C)/7B8 Tg T cell transfer), marble burying index, time spent in the center of an open field, and % interaction and total distance traveled during the sociability test of TCRα KO (b) or IL-17a KO (c) offspring (n=12/15 (TCRa;poly(I:C)/7B8 Tg T cell transfer); n=12/14 (IL-17a KO;poly(I:C)/7B8 Tg T cell transfer). Data pooled from 2-3 independent experiments. d-e, Representative SATB2 staining in the cortex of the animals prepared as in (a). Arrows indicate cortical patches. Scale bar, 100 μm. f-g, Quantification of SATB2⁺ cells (n=7/6 (TCRα KO;poly(I:C)/7B8 Tg T cell transfer); n=6/7 (IL-17a KO;poly(I:C)/7B8 Tg T cell transfer). h, Cortical patch size (n=5/5 (TCRα KO;poly(I:C)/7B8 Tg T cell transfer); n=4/4 (IL-17a KO;poly(I:C)/7B8 Tg T cell transfer). i-j, IL-17a concentrations in maternal plasma collected at E14.5. k, Flow cytometry of ileal CD4⁺ T cells (gated on CD4⁺TCR-β⁺) stained intracellularly for IL-17a. Mononuclear cells were collected from small intestines of poly(I:C)-treated IL-17a KO mothers transferred with 7B8 Tg CD4⁺ T cells. CD45.1⁺ cells refer to donor cells and CD45.2⁺ to recipient cells. *p<0.05, **p<0.01, ***p<0.001, ****p<0.0001 as calculated by Student's t-test (b-c,h-j) and one-way (f-g) ANOVA with Sidak post-hoc tests. Graphs indicate mean+/−s.e.m.

FIG. 12. A mix of twenty human commensals induces colonic Th17 cell differentiation in SFB-absent Jax mice. a, Schematic of the experimental design. b, Flow cytometry of CD4⁺ T cells (gated on CD4⁺TCR-β⁺) stained intracellularly for IL-17a and RORγt. Mononuclear cells were collected from colons of poly(I:C)-treated Jax mothers with/without human bacteria-gavage. c, Representative SEM images of epithelial surfaces in the ilea from 2 independent experiments. d-e, Total interaction time (d), and total distance traveled (e) during the sociability test of adult offspring described in (a) (n=23/22/13 for vehicle-gavaged only/human bacteria-gavaged+isotype control antibody/human bacteria-gavaged+anti-IL-17a antibody; 4 independent experiments). f-g, Quantification of SATB2⁺ cells (n=5/group) and cortical patch size (n=7/6/5 (poly(I:C);vehicle-treated Jax/human bacteria-gavaged Jax with isotype control antibody/human bacteria-gavaged Jax with anti-IL-17a antibody). *p<0.05 as calculated by one-way (d, e, g) and two-way (f) ANOVA with Tukey post-hoc test. Graphs indicate mean+/−s.e.m.

FIG. 13. The IL-17a pathway promotes abnormal behavioral phenotypes in MIA offspring born to mice colonized with human commensal bacteria. a, Schematic representation of the experimental design. b, Quantification of bacterial colonization levels through colony forming unit (CFU) counts or qPCR analyses. c, USV index (n=13/12/28/16/17/14 (poly(I:C);vehicle/SFB/Listeria monocytogenes/Bacteroides fragilis/Bifidobacterium adolescentis/CD-SpA 2A). d-e, Maternal plasma concentrations of IL-17a/IFN-γ at E14.5 (n=4/4/3/6/3 (poly(I:C);vehicle/Listeria monocytogenes/Bacteroides fragilis/Bifidobacterium adolescentis/CD-SpA 2A). f, qPCR analysis measuring relative SFB levels in Jax mice gavaged with various bacteria; from two independent experiments. *p<0.05, ****p<0.0001 as calculated by one-way (c-f) ANOVA with Tukey post-hoc tests and Student's t-test (b). N.D., not determined. N.S., not significant. Graphs indicate mean+/−s.e.m.

FIG. 14. Table 1 provides examples of Th17 cell biogenesis-promoting bacteria.

FIG. 15. Table 2 provides a representation of social behavior testing in mice (See Example 1).

FIG. 16. Table 3 provides a listing of PCR primers used in Example 1.

### DETAILED DESCRIPTION OF THE DISCLOSURE

The present disclosure describes the finding that the presence of maternal gut bacteria that promote biogenesis of T helper 17 (Th17) cells in the maternal intestine increases the risk of an offspring developing developmental disorders including neurodevelopmental disorders. For example, in animal models it was observed that exposure of mothers, who had gut bacteria that promote biogenesis of Th17 cells, to simulated viral infections had a higher incidence of offsprings developing neurodevelopmental disorders.

The term “gut bacteria” or “bacteria in the gut” means one or more types of bacteria that are identified as being resident in the gastrointestinal (GI) tract. The presence of gut bacteria may be determined by obtaining a sample of its contents (e.g., contents of the GI tract) or tissue, or may be inferred from their presence in excrements such as fecal material.

The neurodevelopmental disorders envisioned in the disclosure include Autistic Spectrum Disorders (ASD) and schizophrenia.

Various embodiments of the method are provided in this disclosure. The methods may refer to female subjects. In one embodiment, the female subject is a human. In one embodiment, the female subject is a non-human animal.

The present disclosure provides a method of identifying if a female is at risk of producing an offspring with a neurodevelopmental disorder (NDD) or if there is a risk of an offspring with NDD being born to a female. The method comprises testing for the presence of gut bacteria that promote Th17 cell biogenesis in the female. In one embodiment, the method comprises testing GI tract content or fecal material from the female for the presence of gut bacteria that promote Th17 cell biogenesis. The term “promotion of biogenesis” or similar terms as used in connection with Th17 cells means both an increase in number of Th17 cells as well as differentiation of existing T cells. Thus reference to gut bacteria that promote Th17 cell biogenesis means the bacteria increase the number as well as the differentiation of the Th17 cells.

The method can be used in a female subject that is pregnant, or in a female subject that is planning to get become pregnant or likely to get pregnant, or that may get pregnant.

The testing of the female subject may be carried out periodically during pregnancy, prior to pregnancy, or shortly (within a few days) after the birth of the offspring to monitor the risk in the female of producing an offspring with NDD, or to assess the risk that a newborn offspring has or will develop NDD. For example, monitoring can be done on a monthly, weekly, or daily basis, or at any desired frequency. In one embodiment, the testing can be carried out for female subjects who previously produced an offspring with NDD, and are likely to have or planning to have additional offspring.

The detection of presence of Th17 cell biogenesis-promoting bacteria may be carried out by detecting the presence of specific antigens or specific polynucleotide sequences associated with the bacteria. For example, specific antigens may be detected by affinity binding techniques including antibody binding. Specific sequences may be detected by using PCR or other sequencing methods. These techniques are well known in the art. The presence of Th17 cell biogenesis-promoting bacteria may additionally or alternatively be determined by functional assays—such as by determining if the bacteria can stimulate the biogenesis of Th17 cells in vitro or in animal models, or if the bacteria can promote other immunological events such as enhancing dendritic cell function, or by the presence of metabolites or byproducts that are selectively produced by such bacteria.

The presence of gut-resident Th17 cell biogenesis-promoting bacteria can be detected in samples of fecal materials obtained from an individual. The presence of gut-resident Th17 cell biogenesis-promoting bacteria may also, or alternatively, be detected by obtaining samples directly from the gut, such as content or tissue. In one embodiment, specific antigens or markers for the particular Th17 cell biogenesis-promoting bacteria may be detected in the female tissue, such as any tissue obtained from the gut, or in circulation, such as in blood (including fractions of blood, such as plasma or serum).

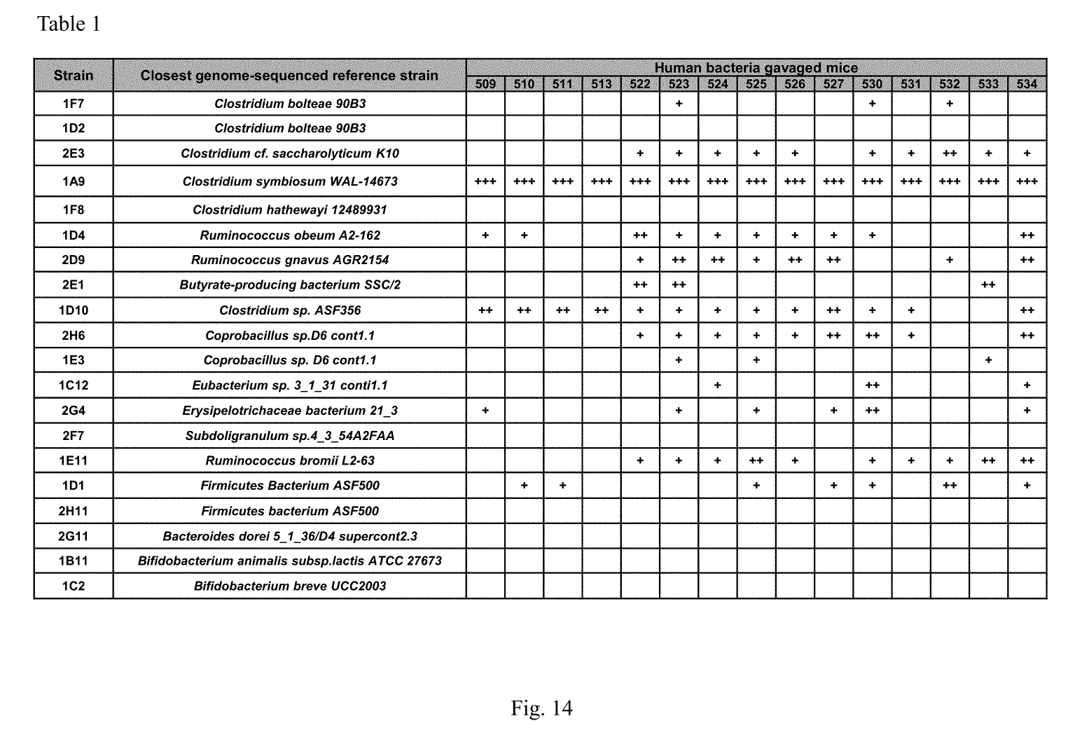

Examples of Th17 cell biogenesis-promoting bacteria include any human bacteria that are known to, or are identified to promote gut Th17 cells. Examples include human bacteria as described in Atarashi et al (Cell, 2015 vol. 163(2) pp 367-380). Examples are also provided in Table 1 (FIG. 14) of present disclosure. Examples include B. adolescentis, twenty human bacteria as described in Atarashi et al (Cell, 2015 vol. 163(2) pp 367-380) and an adherent E. coli isolate, CD-SpA 2A. Examples include strains of Clostridium, Coprobacillus, Eubacterium, Erysipelotrichaceae, Firmicutes, Subdoligranulum, Butyrate producing bacterium, Bifidobacterium, Ruminococcus, and/or Bacteroides.

The present disclosure also provides a method of identification or design of a treatment plan for reducing the risk of NDD in an offspring. The method comprises identifying if a female subject is at risk of producing an offspring that has NDD by detecting for the presence of Th17 cell biogenesis-promoting bacteria in the female gut. If a risk is identified based on such presence, a screening can be carried out to identify suitable agents for inhibiting the bacteria. For example, fecal material can be obtained from the female subject and tested for the presence of Th17 cell biogenesis-promoting bacteria, and if such presence is detected, the identified bacteria (either from the female subject source or elsewhere) can be used to test various anti-bacterial agents, including antibiotics. The bacteria can also be used to screen for other agents or bacteria that antagonize their activities or survival in vitro or in vivo using suitable animal models. Based on identification of suitable anti-bacterial agents, a treatment plan for NDD can be designed. The treatment plan may be implemented for a pregnant female or may be implemented in a female who is planning to become pregnant, who is likely to get pregnant, or who may get pregnant.

The present disclosure provides a method of reducing the risk of a female subject producing an individual with NDD. The method comprises selecting an individual who is at risk of producing an offspring with NDD based on the presence in the maternal gut of Th17 cell biogenesis-promoting bacteria (e.g., as detected in fecal matter), and administering to the female subject an agent or agents that inhibit such bacteria and/or that suppress Th17 cell activity in maternal guts. Administration to the female subject of agents that inhibit Th17 cell differentiation activity may be carried out when the offspring is in utero, or may be carried out before the female subject becomes pregnant. Suitable anti-bacterial agents and/or Th17 cell activity suppressors can be administered at doses and frequencies and via modes of administration that are known in the art for those agents.

An example of a suitable anti-bacterial agent is an antibiotic. Any antibiotic that is known to inhibit, or is determined to inhibit the growth of Th17 cell biogenesis-promoting bacteria, particularly in the gut, can be used. Examples include broad-spectrum antibiotics, or specific antibiotics, including, but not limited to vancomycin, ampicillin, metronidazole, amoxicillin, levofloxacin, gatifloxacillin, streptomycin, tetracycline, chloramphenicol. Thus, one or more antibiotics may be administered to a female who may be diagnosed as having Th17 cell biogenesis-promoting bacteria in the gut or who may be at risk of having Th17 cell biogenesis-promoting bacteria in the gut. Other suitable anti-bacterial approaches may include protein therapy (antibody specific to bacteria), probiotics (antagonizing bacteria), prebiotics (diet), small molecules and bacteriophages.

An effective amount of the antibiotic or antibiotics and/or other therapies disclosed herein can be administered to a subject in need thereof. The term “effective amount” as used herein is the amount sufficient to achieve, in a single or multiple doses, irrespective of the mode of administration, the intended purpose of treatment. The exact amount desired or required will vary depending on the mode of administration, patient specifics and the like. Appropriate effective amounts can be determined by one of ordinary skill in the art (such as a clinician or other similar medical care providers) with the benefit of the present disclosure.

Because Th17 activity is known to include an increase in circulating IL-17a levels, in one embodiment, the method comprises: a) based on the presence of gut bacteria that promote Th17 cell biogenesis, identifying pregnant mothers with a risk of producing offsprings with NDD; and b) treating the mother with inhibitors of IL-17a or its related cytokines IL-17f activity.

The above methods for identifying a risk of a mother producing an offspring with NDD are applicable to not only pregnant women, but can also be carried out in women who are not pregnant but may be planning to get pregnant. For example, the present methods can be carried out in a woman who is planning to get pregnant. In one embodiment, the methods can be carried out in a female subject who has previously produced an offspring who has NDD.

The screening method to identify the presence of Th17 cell biogenesis-promoting bacteria in the gut of women is contemplated herein as a pre-pregnancy screen, or as a monitoring screen during pregnancy. Once risk is identified, treatment approaches can be identified and administered to the female subject. The goal of the treatment approaches is to eliminate, minimize or reduce the Th17 cell biogenesis-promoting bacteria in the maternal gut. In one embodiment, once risk is identified, treatment approaches that are suitable, can also be implemented for the offspring in utero.

Examples of compounds that inhibit Th17 activity include inhibitors of RORγt activity and/or IL-17 activity. (See U.S. application Ser. No. 15/042,976, incorporated herein by reference) Exemplary inhibitors of RORγt activity include: TMP778 (Skepner et al. 2014, J Immunol 192:2564-2575), SR1001 (Solt et al. 2011, Nature 472:491), SR1555 (Solt et al. 2012, ACS Chem Biol 7:1515), and SR2211 (Kumar et al. 2012, ACS Chem Biol 7:672). These and other inhibitors of RORγt activity, as well as assays for detecting/assessing RORγt activity, are described in, for example, U.S. Patent Application Publication Nos. 2014/0275490, 2013/0085162, 2013/0065842 and 2007/0154487; U.S. Pat. No. 9,101,600; and WO2013/036912, WO2012/074547, WO2013/079223, WO2013/178362, WO2011/112263 (SR-9805), WO2011/112264, WO2010/049144, WO2012/027965, WO2012/028100, WO2012/100732, WO2012/100734, WO2011/107248, WO2012/139775, WO2012/064744, WO2012/106995, WO2012/147916, and WO2010/049144, the relevant disclosures of each of which are incorporated herein by reference. Other inhibitors of RORγt activity are also described in Skepner et al. (2014, J Immunol 192:2564-2575), Skepner et al. (2015, Immunology, July; 145(3):347-56), Nishiyama et al. (2014, Bioorganic & Medicinal Chemistry 22:2799-2808; compound 5b), Fauber et al. (2014, J Medicinal Chem 57:5871-5892), Liu et al. (2014 J Immunol 192:59-72), Mele et al. (2013, J Exp Med 210:2181-2190), Dhar et al. (2013, Annual Reports in Medicinal Chemistry 48:169-182), Xu et al. (2011, J Biol Chem 286:22707; ursolic acid) and Huh et al. (2011, Nature 472:486-490), the relevant disclosures of each of which are incorporated herein by reference.

Exemplary inhibitors of IL-17 activity include antibodies specific for IL-17a or the IL-17 receptor (IL-17R). An example of an inhibitor of IL-17 activity is a human monoclonal antibody or a humanized monoclonal antibody. Such antibodies can block IL-17R engagement by IL-17A. The human monoclonal antibody can be brodalumab (AMG 827), which is specific for the IL-17R, or ixekizumab (LY2439821) or secukinumab (AIN457), which are specific for IL-17A. Antibodies specific for the p19 subunit of IL-23 or the p40 subunit of IL-23 and IL-12 may also be used. Exemplary antibodies specific for the p19 subunit of IL-23 include MK-3222 (SCH 900222), CNTO 1959, and AMG 139. Exemplary antibodies specific for the p40 subunit of IL-23 and IL-12 include Stelara (ustekinumab; CNTO 1275). Exemplary anti-IL17 monoclonal antibodies are described in, for example, Garber et al. (2012, Nature Biotechnology 30:475-477), the relevant disclosure of which is incorporated herein by reference.

Agents/compounds described above are available commercially as follows: brodalumab (AMG 827) and AMG 139 are available from Amgen/Medlmmune; ixekizumab (LY2439821) is available from Eli Lilly; secukinumab (AIN457) is available from Novartis; MK-3222 (SCH 900222) is available from Merck; CNTO 1959 and Stelara (ustekinumab; CNTO 1275) are available from Janssen Biotech (J & J).

Also envisioned for use in methods described herein are antibodies for other Th17 cell specific cytokines, such as, but not limited to IL-17f and IL-22. Antibodies and reagents specific for Th17 specific cell surface proteins, of which CCR6 is an example, are also envisioned for use in methods described herein. See also Hedrick et al. (2010, Expert Opin Ther Targets 14:911-922), the entire content of which is incorporated herein by reference). Blocking antibodies specific for IL-23 receptor (IL-23R) are also envisioned for use in methods described herein. See, for example, US 2014/0275490, which is incorporated herein in its entirety by reference.

Also envisioned herein are antibody fragments or altered/mutated antibodies, particularly those wherein the Fc domain is absent or altered/mutated such that the antibody fragment or mutated antibody can no longer bind to Fc receptors. Also encompassed herein are mutated antibodies or fragments thereof having enhanced binding for MHC Class I related receptor FcRn (Fc receptor neonatal) and/or FcγRIII. FcRn has been shown to be essential for transplacental passage of immunoglobulin G (IgG) antibodies. Methods for generating antibody fragments or mutated antibodies that can have enhanced binding for FcRn or that no longer bind to Fc receptors are described in Firan et al. (2001, Intern Immunol 13:993-1002), the relevant disclosure of which is incorporated herein by reference.

One skilled in the art can readily determine or assess the suitability of other compounds for use in the invention by screening in cellular assays of Th17 activity such as those described herein or known in the art, or in animal models of disease in which Th17 cell activity is implicated such as those described herein and elsewhere. See, for example, U.S Patent Application Publication No. 2007/0154487, the relevant disclosure of which is incorporated herein by reference.

In one embodiment, this disclosure provides a method of treating the offspring who is afflicted with or at risk for developing NDD based on being born to a female who has gut bacteria that promote Th17 cell biogenesis. The method comprises treating the offspring that is in utero or no longer in utero by administering pharmacological agents such as, for example, GABAergic receptor agonists or implementing optogenetic tools or chemogenetic tools to correct dysregulated neuronal excitation/inhibition (E/I) ratios in cortical patches of the offspring wherein the E/I ratio is dysregulated. See U.S. patent application Ser. No. 15/416,238, incorporated herein by reference. The pharmacological agents and/or optogenetic tools can be administered to the cortex of the subject to at least partially restore a normal E/I ratio (comparable to E/I ratio in individuals of comparable age who are not afflicted with the same NDD) in patches of cortical malformation wherein the E/I ratio is dysregulated in the absence of treatment. In an embodiment, the pharmacological agents and/or optogenetic tools are administered to and/or in the vicinity of the dysgranular zone of the primary somatosensory cortex (S1DZ) of the subject. In one embodiment, the pharmacological agents and/or optogenetic tools are targeted specifically to patches of cortical malformation wherein the E/I ratio is dysregulated. The patches of cortical malformation may be located in the S1DZ. The pharmacological agents and/or optogenetic or chemogenetic tools may be administered or used alone or in conjunction with agents that modulate (promote or inhibit) IL-17 activity or interferon-gamma (IFN-γ) activity. Agents that promote Interleukin-17 (IL-17) activity include exogenous IL-17a and/or IL-17f (e.g., synthetic/manmade/recombinant IL-17a or IL-17f) and IL-17 receptor (IL-17R) agonists. Inhibitors of IL-17 activity include, e.g., antagonistic IL-17 antibodies and/or inhibitors of IL-17, IL-17R, or RORγt activity (e.g., small molecule inhibitors) and IL-17R antagonists. Agents that promote IFN-γ activity include exogenous IFN-γ (e.g., synthetic/manmade/recombinant IFN-γ) and IFN-γ receptor (IFN-γR) agonists. Agents that inhibit IFN-γ activity include, e.g., antagonistic IFN-γ antibodies, inhibitors of IFN-γ activity, small molecule inhibitors of IFN-γ, and IFN-γR antagonists.

In an embodiment, this disclosure provides a method of identifying risk of a female subject producing an offspring with neurodevelopmental disorder comprising identifying in the gut of the female subject (gut-resident bacteria), the presence of one or more types of bacteria that promote Th17 cell biogenesis, wherein the presence of one of more types of such bacteria indicates a risk that the female subject will produce an offspring with a developmental disorder. The presence of gut-resident bacteria may be determined by determining their presence in fecal material or by obtaining a sample of food material in the gut or tissue sample. The female subject may be pregnant with the offspring, or may be planning to get pregnant, or is not pregnant. The presence of one or more types of bacteria may be detected monthly, weekly, or daily during the pregnancy. The female subject may or may not have previously produced an offspring who is afflicted with a neurodevelopmental disorder, such as, for example, an autism spectrum disorder (ASD). The bacteria that promote Th17 cell biogenesis may comprise one or more bacteria listed in Table 1 (FIG. 14), B. adolescentis or an E. coli isolate, CD-SpA 2A. In one embodiment, the bacteria that promote Th17 cell biogenesis can comprise a strain of bacteria from the species Clostridium, Coprobacillus, Eubacterium, Erysipelotrichaceae, Firmicutes, Subdoligranulum, Butyrate producing bacterium, Bifidobacterium, Ruminococcus, and/or Bacteroides, or combinations thereof. The presence of the bacteria can be determined by immunological techniques to detect specific antigens or nucleic acid amplification techniques to detect specific polynucleotide sequences.

In an embodiment, this disclosure provides a method of identifying an individualized treatment plan for a female subject to reduce the risk of the female subject producing an offspring with a neurodevelopmental disorder comprising screening the type of bacteria present in the gut of the female subject, identified as promoting, Th17 cell biogenesis, for: i) effective anti-bacterial agents, and/or ii) effective Th17 cell suppressing agents.

In an embodiment, this disclosure provides a method of identifying an individualized treatment plan for a female subject to reduce the risk of the female subject producing an offspring with a neurodevelopmental disorder comprising a) identifying in the gut of the female subject, the presence of one or more types of bacteria that promote Th17 cell biogenesis, wherein the presence of one of more of the said bacteria indicates an increased likelihood that the female subject will produce an offspring with a neurodevelopmental disorder; and b) if the presence of said bacteria is determined, then i) screening the bacteria for effective anti-bacterial agents, and/or screening for ii) effective Th17 cell suppressing agents, wherein the effective anti-bacterial agents inhibit the growth of the one or more bacteria that promote Th17 cell biogenesis, and the effective Th17 cell suppressing agents inhibit the number or activity of the gut Th17 cells.

In an embodiment, this disclosure provides a method of reducing the risk of a female subject producing an offspring having a neurodevelopmental disorder comprising testing for the presence of bacteria that promote Th17 cell biogenesis in a fecal sample obtained from the female subject, and if the presence of such bacteria is detected, administering to the female subject, a therapy that inhibits the bacteria, reduces Th17 cell biogenesis promoting activity of said bacteria, and/or reduces the activity of the Th17 cells. The method may further comprise testing the bacteria for suitable anti-bacterial agent or agents that inhibit Th17 cell biogenesis activity of the bacteria, and then administering the identified agent or agents to the female subject. The agent may be an antibiotic. The antibiotic may be a broad spectrum antibiotic. Examples of antibiotics include, but are not limited to, vancomycin, ampicillin or metronidazole, amoxicillin, levofloxacin, gatifloxacillin, streptomycin, tetracycline, chloramphenicol. In an embodiment, the agent may be an inhibitor of Th-17 activity or IL-17 activity.

In an embodiment, this disclosure provides a method of reducing the risk of a female subject producing an offspring having a neurodevelopmental disorder comprising: a) obtaining a fecal sample from the female subject; b) testing the fecal sample for the presence of bacteria that promote Th17 cell biogenesis; and c) if the presence of such bacteria is detected, administering to the female subject, a therapy that inhibits the growth of the bacteria, reduces the Th17 cell biogenesis promoting activity of said bacteria, and/or reduces the activity of Th17 cells. The therapy that inhibits the bacteria can comprise administering a composition comprising an antibiotic, which may be a broad spectrum antibiotic. Examples of antibiotics include, but not limited to, vancomycin, ampicillin or metronidazole, amoxicillin, levofloxacin, gatifloxacillin, streptomycin, tetracycline, chloramphenicol.

The methods provided in this disclosure may be carried out prior to pregnancy or during pregnancy, or if the treatment is administered to the offspring, it may be carried out after birth.

In an embodiment, this disclosure provides a method of reducing the risk of an offspring having a neurodevelopmental disorder comprising: testing for the presence of bacteria that promote Th17 cell biogenesis in a fecal sample obtained from the mother; and if the presence of such bacteria is detected, administering to the offspring a therapy comprising a pharmacological agent or implementing optogenetic or chemogenetic tools that corrects dysregulated neuronal excitation/inhibition (E/I) ratios in the cortex of the offspring. The pharmacological agent can be a GABAergic receptor agonist. The GABAergic receptor agonist can be a synthetic GABA, muscimol, a barbiturate, or a benzodiazapine. The optogenetic tools can be channelrhodopsin, halorhodopsin, or an OptoXR.

The following examples are provided as illustrative of the present methods. These examples are not intended to be restrictive in any way.

### Example 1

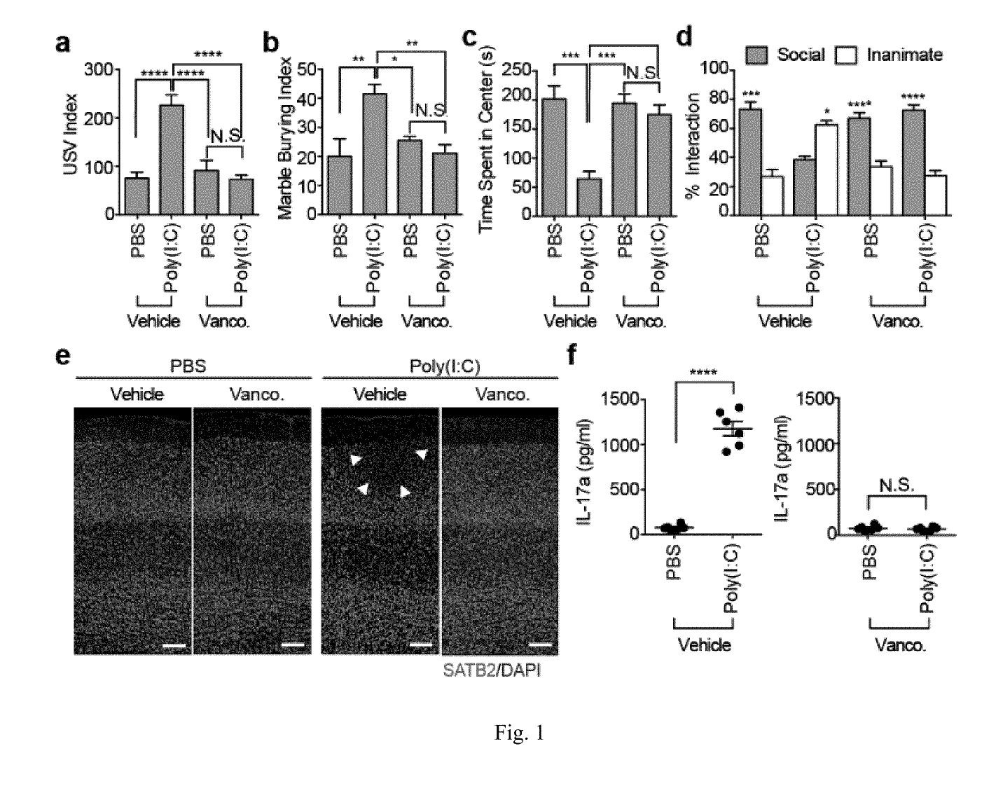

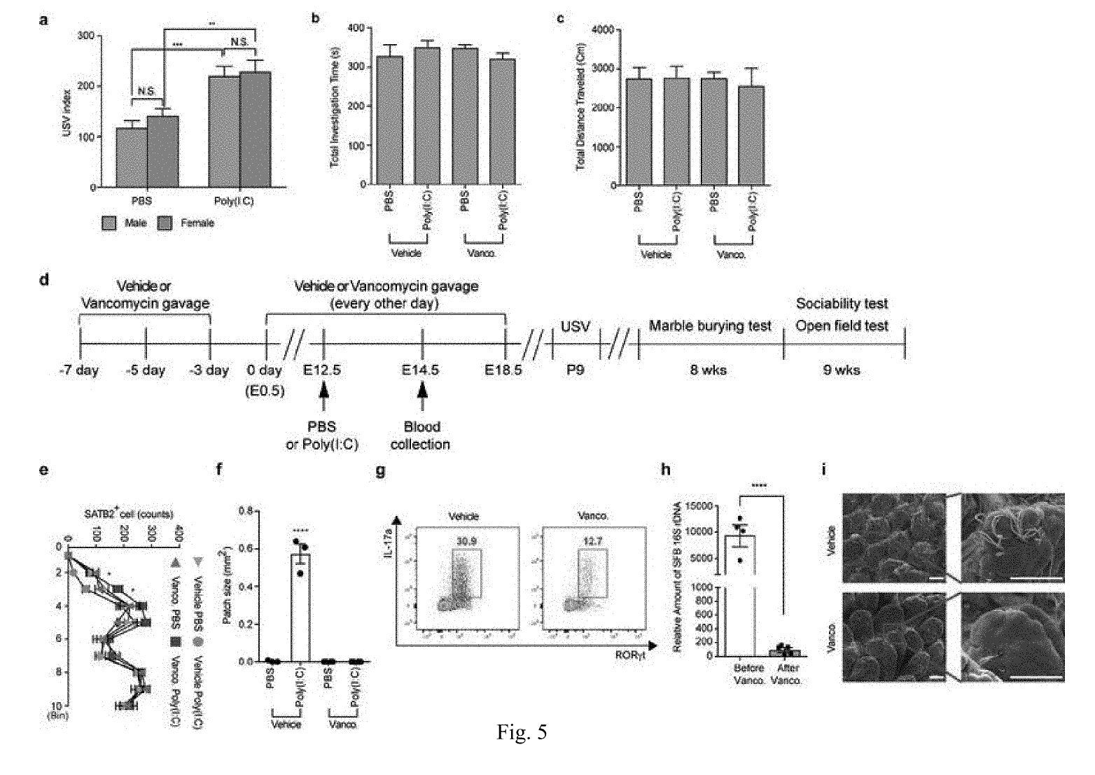

Pups from mothers injected with poly(I:C) at embryonic day 12.5 (E12.5) emit more ultrasonic vocalization (USV) calls than those from PBS-injected mothers (FIG. 1a). Unlike other behavioral phenotypes that are often more strongly manifested in male than in female offspring, USV calls were enhanced in both sexes among MIA offspring (FIG. 5a). In addition, fetal exposure to MIA led to other behavioral abnormalities including enhanced repetitive behaviors (increased marble burying), increased anxiety (decreased time spent in the center of an open field arena) and social interaction deficits (decreased interaction with a social stimulus) in adult male offspring (FIG. 1b-d). These behavioral phenotypes did not emerge from changes in activity or arousal levels as the total investigation time and the total distance traveled during the sociability test remained comparable (FIGS. 5b and c). To investigate whether maternal commensal bacteria influence MIA-associated behaviors, we treated C57BL/6 wildtype (WT) mice from our vivarium with the broad spectrum antibiotic vancomycin prior to phosphate-buffered saline (PBS) or poly(I:C) administration (FIG. 5d). Interestingly, pre-treating poly(I:C)-injected mothers with vancomycin prevented development of all four behavioral abnormalities in MIA offspring (FIG. 1a-d).

We previously showed that MIA offspring exhibit cortical patches devoid of cortical layer-specific markers, such as SATB2 (Choi et al., Science 351, 933-939, (2016)), and these cortical patches resemble lesions described in brains of ASD patients (Casanova et al. Acta neuropathologica communications 1, 67, (2013); Stoner et al. The New England journal of medicine 370, 1209-1219, (2014)). These cortical patches are predominantly localized in the area encompassing the dysgranular zone of the primary somatosensory cortex (S1DZ) and are closely associated with the MIA-associated behavioral abnormalities (Yim et al., co-submitted manuscript). Unlike the adult offspring derived from poly(I:C)-injected dams, the offspring of poly(I:C)-injected mothers pre-treated with vancomycin failed to develop cortical patches (FIG. 1e and FIG. 5e, f). Vancomycin treatment of poly(I:C)-injected pregnant dams led to a decrease in the proportion of Th17 cells in the small intestine with a concomitant reduction in the levels of IL-17a in the maternal plasma, compared to those of the control group (FIG. 1f and FIG. 5g). These data indicate that the presence in pregnant mice of commensal bacteria sensitive to vancomycin is crucial for the induction of MIA-associated behavioral and brain abnormalities in the offspring. Furthermore, the presence of such bacteria is associated with increased proportion of Th17 cells in the small intestines and high levels of IL-17a in the plasma of poly(I:C)-treated pregnant dams.

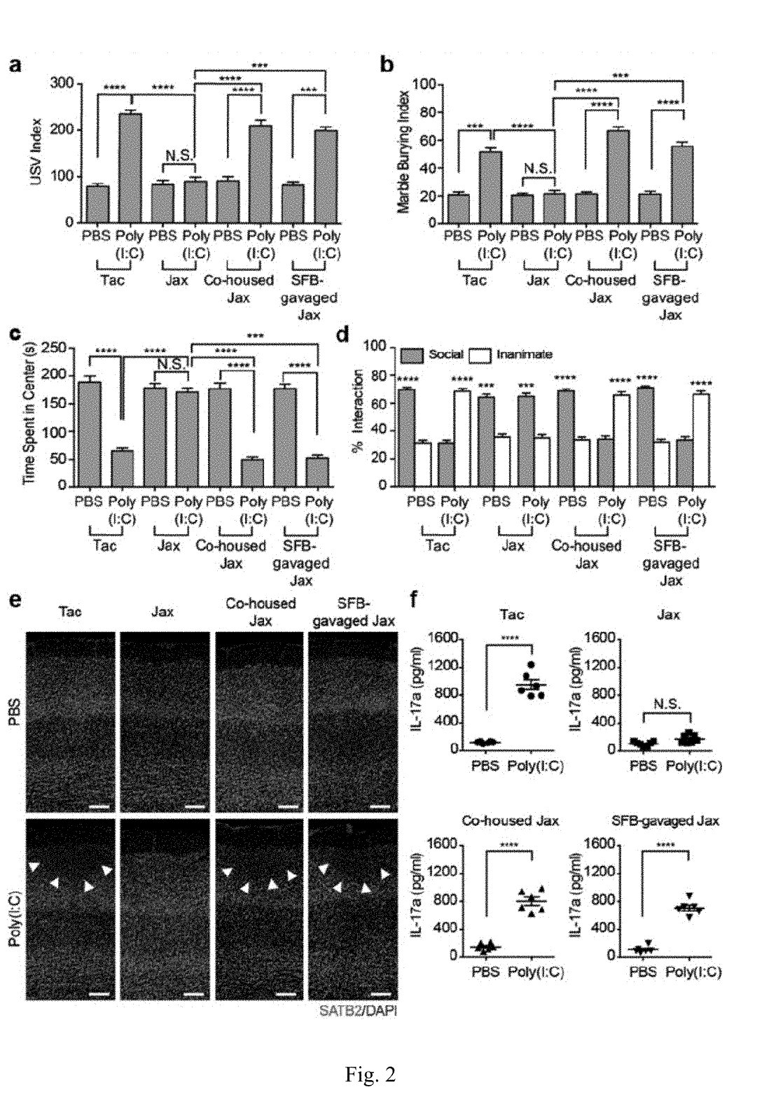

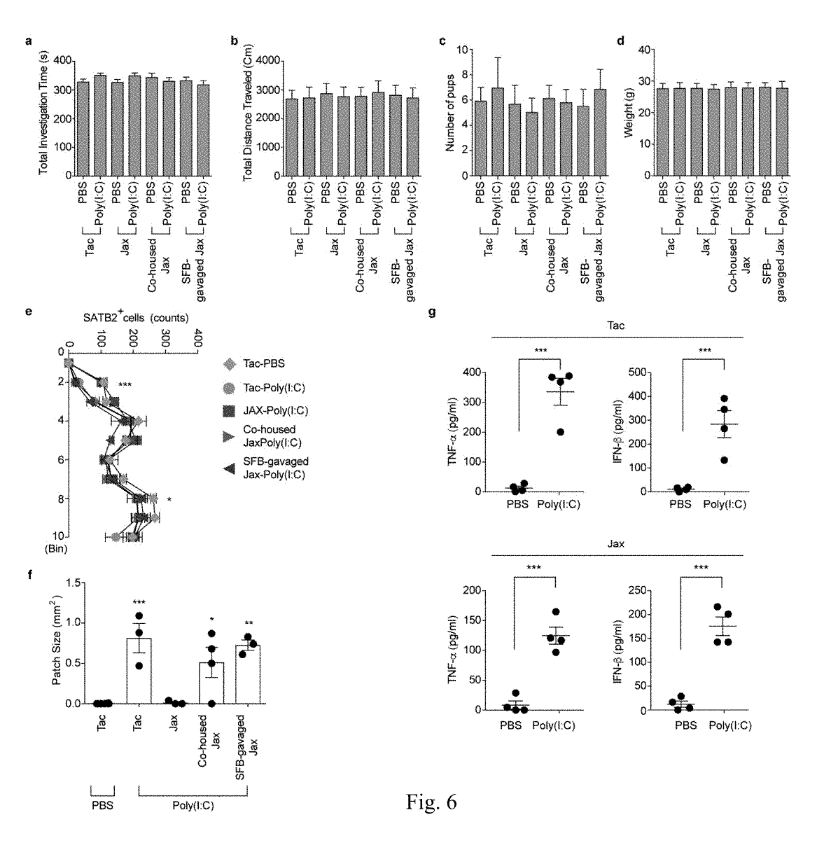

Among commensal bacteria in laboratory mice, SFB is susceptible to vancomycin and contributes disproportionately to Th17 cell biogenesis in the small intestine. Indeed, qPCR analyses of mouse fecal samples showed that intestinal colonization by SFB is severely reduced upon vancomycin treatment (FIG. 5h). We also performed scanning electron microscopy (SEM) to visualize SFB, which is associated with intestinal epithelial cells (IEC). Whereas plenty of SFB were found attached to the ileal mucosa of the PBS-treated dams, IEC-associated SFB were not detected in the vancomycin-treated dams (FIG. 5i). We therefore next investigated if the presence of SFB in pregnant mice correlated with the MIA-associated behavioral phenotypes in offspring. C57BL/6 mice from Taconic Biosciences (Tac) have abundant Th17 cells in their small intestine due to the presence of SFB; in contrast, C57BL/6 mice from Jackson Laboratories (Jax), which lack SFB, have few intestinal Th17 cells. Unlike offspring from poly(I:C)-injected Tac mothers, those from poly(I:C)-injected Jax mothers failed to show any of the MIA-associated behavioral phenotypes (FIG. 2a-d and FIG. 6a, b). Poly(I:C)-treated Tac versus Jax mothers had litters of similar size and the pups had similar weights (FIG. 6c, d). Sizes of the cortical patches observed in the offspring of poly(I:C)-injected Tac mothers were highly correlated with the severity of the MIA-associated behavioral abnormalities (Yim et al., co-submitted manuscript). Consistent with this finding, MIA offspring from the SFB-deficient Jax mothers injected with poly(I:C) had no cortical abnormalities, as assessed by SATB2 staining (FIG. 2e and FIG. 6e, f). Unlike in Tac mice, Jax mothers injected with poly(I:C) did not show systemic increases in IL-17a in the plasma (FIG. 2f). However, poly(I:C) injection of both Tac and Jax animals resulted in the robust induction of TNF-α and IFN-β, compared to PBS control mice (FIG. 6g).

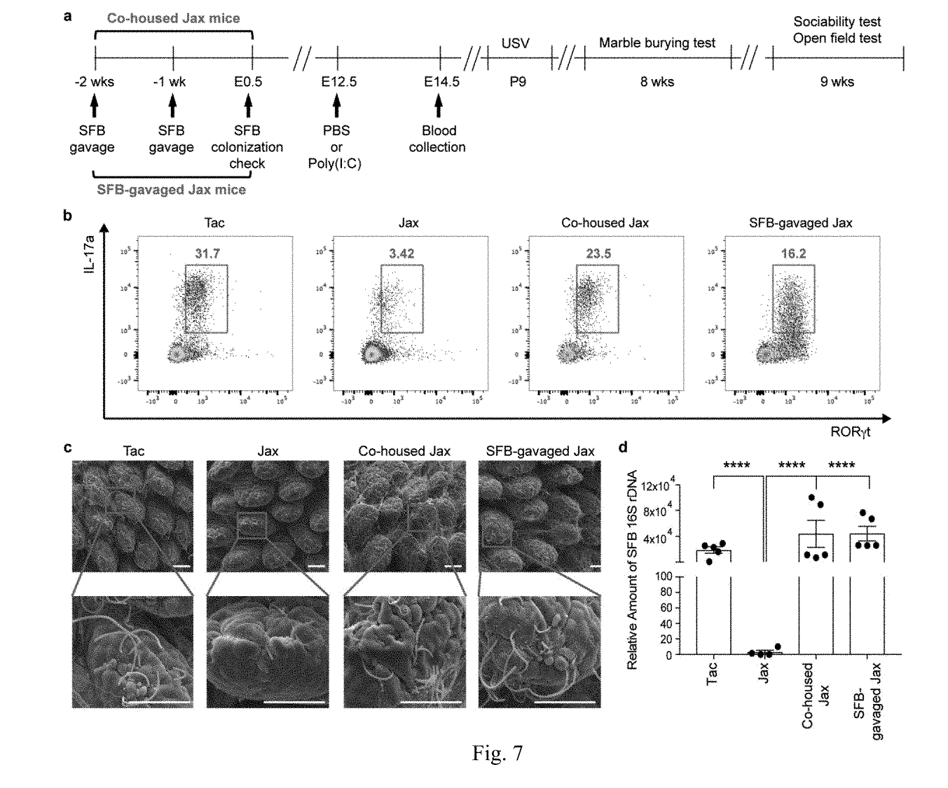

Offspring of poly(I:C)-injected Jax mothers that had been either co-housed with Tac mice or gavaged with a fecal slurry from SFB mono-colonized mice (FIG. 7a) displayed MIA-associated behavioral and cortical abnormalities (FIG. 2a-e). These MIA-associated phenotypes in the offspring from both the co-housed as well as SFB-gavaged Jax mothers were accompanied by increased proportion of gut-residing Th17 cells, consistent with the presence of SFB in the small intestine of these mice (FIG. 7b-d). Accordingly, the co-housed and SFB-gavaged Jax mothers exhibited increased levels of plasma IL-17a following poly(I:C) injections (FIG. 2f). Thus, the presence or absence of a single commensal bacterial species SFB in the intestines of pregnant mothers influences long-lasting behavioral and neurodevelopmental outcomes in the offspring exposed to MIA.

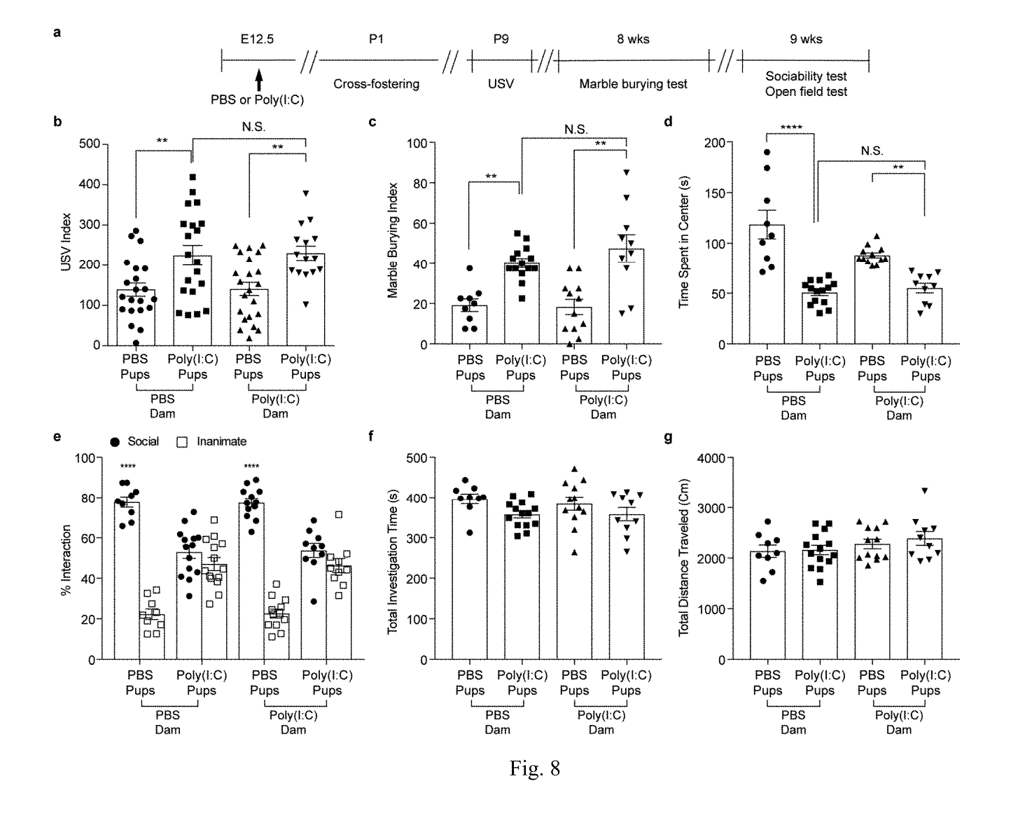

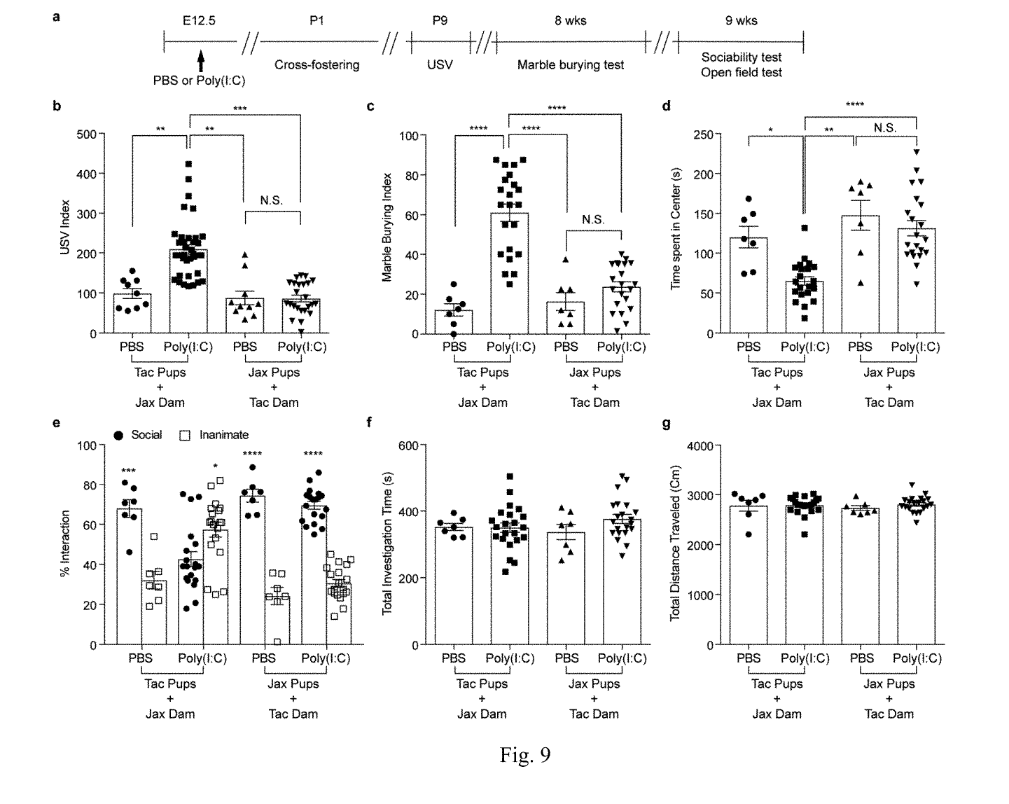

We next investigated if maternal exposure to poly(I:C)-induced inflammation or maternal colonization with SFB influence MIA-associated behaviors in offspring after birth. We performed cross-fostering experiments by switching newborns between PBS- and poly(I:C)-treated Tac mothers or between SFB-positive Tac and SFB-negative Jax mothers (FIGS. 8a and 9a). Whereas offspring derived from poly(I:C)-injected mothers, but reared by PBS-injected mothers, exhibited behavioral abnormalities, those from PBS-injected mothers that were reared by poly(I:C) mothers exhibited normal behaviors (FIG. 8b-g). Likewise, offspring derived from poly(I:C)-injected Tac mothers, but reared by Jax mice, exhibited behavioral abnormalities, whereas those derived from poly(I:C)-injected Jax mothers and reared by Tac mothers displayed normal behaviors (FIG. 9b-g). These data indicate that the presence of SFB in the small intestine of mothers as well as the immunological effects of poly(I:C) are critical during pregnancy, not during post-natal nursing, for licensing MIA-induced behavioral abnormalities in offspring.

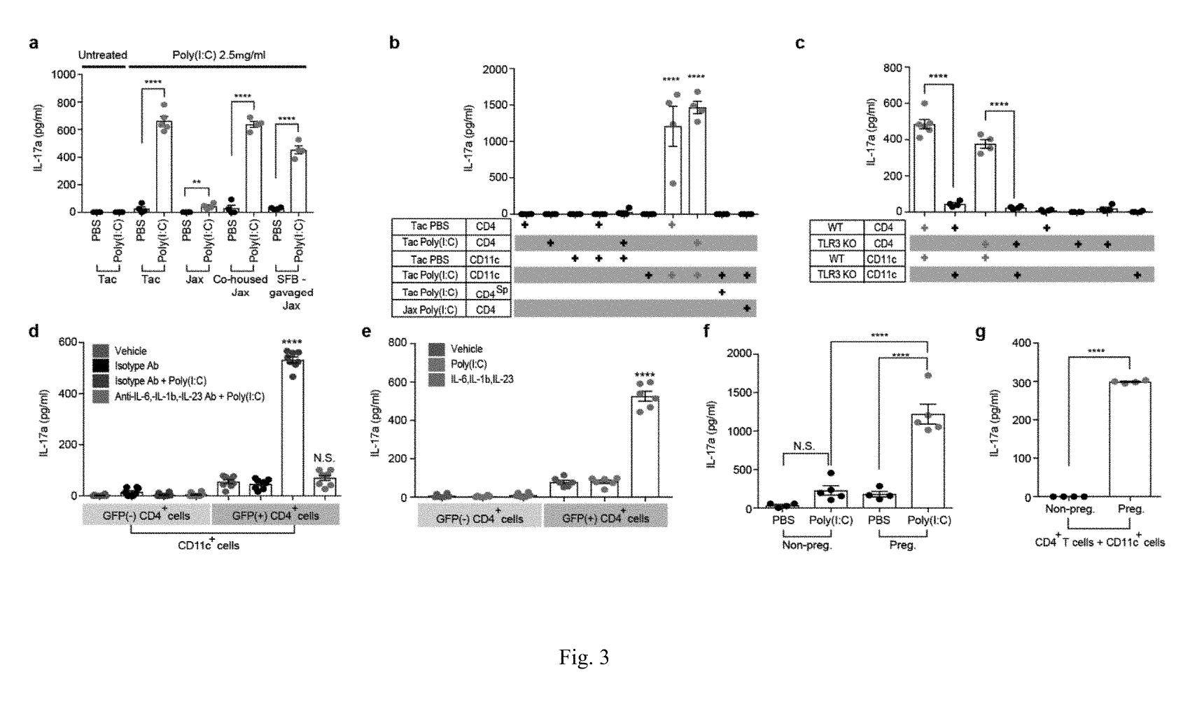

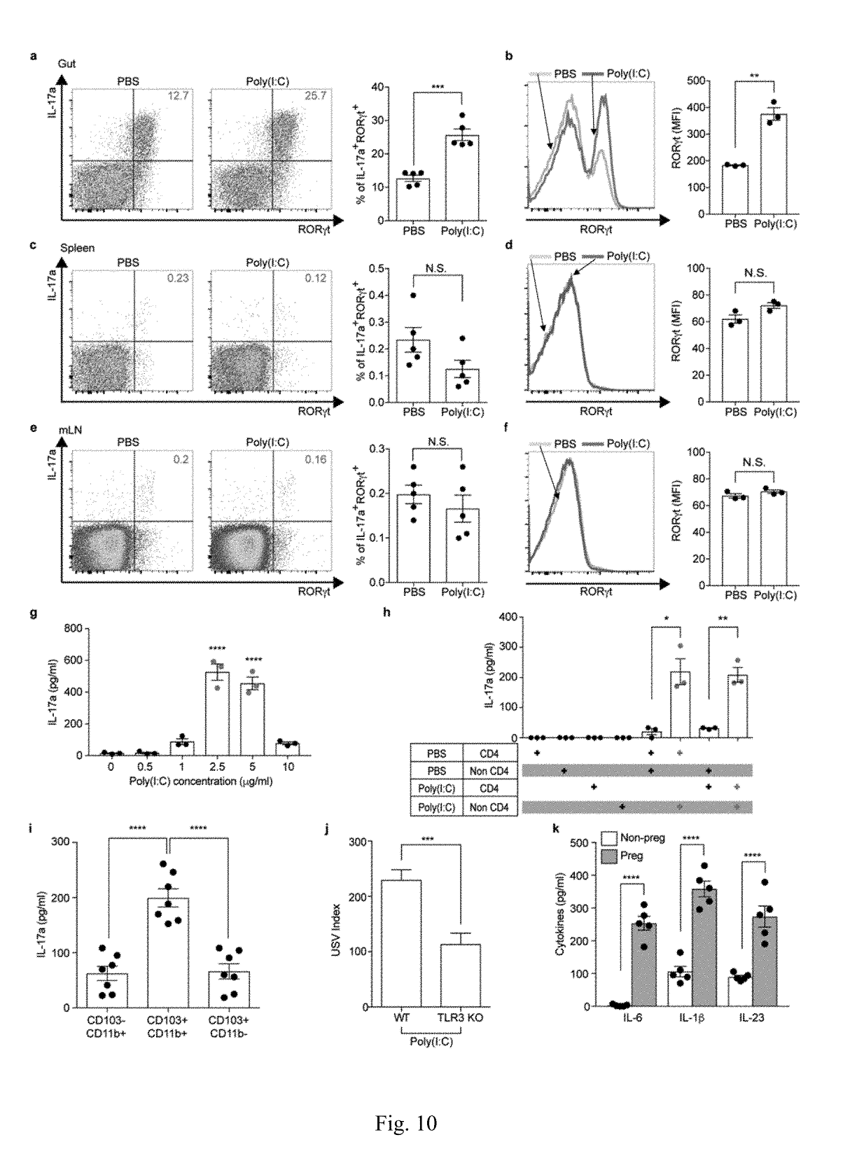

MIA leads to an increase in plasma IL-17a levels in pregnant mice as early as 12-24 h following E12.5 poly(I:C) injection. Such a rapid increase strongly suggested that pre-existing Th17 cells, rather than de novo differentiating Th17 cells, are the major source for IL-17a in pregnant mice exposed to inflammation. As Th17 cells are most abundant in the small intestine lamina propria, we next investigated whether poly(I:C) stimulates IL-17a production via gut-residing Th17 cells. In poly(I:C)-treated pregnant mice, T cells isolated from lamina propria, but not spleen or mesenteric lymph node, expressed high levels of IL-17a and had increased RORγt expression as compared to cells from PBS-treated mice (FIG. 10a-f). Consistent with these observations, ileum-associated mononuclear cells, isolated from poly(I:C)-injected Tac pregnant mice and further stimulated in vitro with poly(I:C), produced higher levels of IL-17a compared to those from PBS-treated Tac mice (FIG. 10g and FIG. 3a). In contrast, mononuclear cells from poly(I:C)-treated Jax mice secreted only small amounts of IL-17a (FIG. 3a). Introduction of SFB into Jax mice either by co-housing them with Tac mice or by gavaging them with an SFB-containing fecal slurry was sufficient to enable ileum-associated mononuclear cells to produce high levels of IL-17a (FIG. 3a). To examine which cells are involved in the poly(I:C) response, we separately isolated CD4⁺ as well as non-CD4⁺ cells from PBS- and poly(I:C)-treated pregnant Tac mice and co-cultured the isolated cells from each experimental group. The non-CD4⁺ fraction derived from the poly(I:C)-, but not from the PBS-treated mothers, promoted IL-17a production when added to cultures containing CD4⁺ cells from either PBS- or poly(I:C)-treated pregnant mice (FIG. 10h). We next tested if poly(I:C)-primed CD11c⁺ DC cells were capable of supporting CD4⁺ T cells to produce IL-17a. Adding CD11c⁺ cells derived from poly(I:C)-, but not from PBS-treated, pregnant mice to ex vivo cultures containing ileal CD4⁺ T cells that were isolated from either PBS- or poly(I:C)-treated pregnant Tac mice led to robust expression of IL-17a (FIG. 3b). In contrast, neither splenic CD4⁺ T cells of poly(I:C)-injected pregnant Tac mice nor ileal CD4⁺ T cells of poly(I:C)-injected pregnant Jax mice produced IL-17a even with the help of CD11c⁺ cells (FIG. 3b). Thus, both CD4⁺ T cells present in the small intestines of Tac mice and poly(I:C)-activated CD11c⁺ cells are required for robust IL-17a induction. Among the multiple phenotypes of gut-residing DCs, CD103⁺CD11b⁺CD11c⁺ cells were robust inducers of IL-17a when co-cultured with ileal CD4⁺ T cells (FIG. 10i).

Because poly(I:C) activates Toll-like receptor 3 (TLR3), we investigated if this receptor is involved in stimulation of IL-17a production. Whereas CD4⁺ T cells, regardless of their tlr3 genotype, produced IL-17a when mixed with WT CD11c⁺ cells, they failed to do so when co-cultured with TLR3-deficient CD11c⁺ DCs (FIG. 3c). In addition, poly(I:C) injection to TLR3 KO pregnant mice failed to induce MIA-associated USV phenotypes in offspring (FIG. 10j). These data suggest that MIA-associated phenotypes require functional TLR3 expression on gut CD11c⁺ DC.

Inflammatory cytokines such as IL-113/IL-6/IL-23 enhance Th17 cell function and differentiation. Consistent with this notion, co-cultures of sorted CD4⁺ and CD11c⁺ DCs that were isolated from the ilea of poly(I:C)-treated gravid Tac mice and incubated with IL-113/IL-6/IL-23 blocking antibodies failed to produce IL-17a, even when supplemented with poly(I:C) (FIG. 3d). In contrast, GFP⁺ Th17 cells, but not GFP⁻ non-Th17 cells, sorted from the ilea of IL-17a-GFP reporter mice, produced high levels of IL-17a in the presence of exogenous IL-113/IL-6/IL-23, even in the absence of poly(I:C)-treated CD11c⁺ cells (FIG. 3e). Collectively, these data indicate that poly(I:C) treatment leads to the activation of gut-residing CD103⁺CD11b⁺CD11c⁺ cells, which stimulate poised Th17 cells to produce IL-17a through secretion of IL-1β/IL-6/IL-23.

Surprisingly, we noted that poly(I:C) injection of non-pregnant females failed to increase the levels of plasma IL-17a (FIG. 3f). Co-culture of ileal CD4⁺ and CD11c⁺ cells isolated from poly(I:C)-treated pregnant females, but not from poly(I:C)-treated non-pregnant females, resulted in secretion of IL-17a ex vivo (FIG. 3g). Consistent with these findings, gut CD11⁺ DCs isolated from poly(I:C)-treated pregnant females, but not from poly(I:C)-treated non-pregnant females, produced increased levels of IL-1β/IL-6/IL-23 (FIG. 10k). In sum, these data collectively suggest that Th17 cell-inducing gut bacteria, a pro-inflammatory stimulus and pregnancy are all required for the systemic increase of IL-17a in maternal plasma, which promotes MIA-associated behavioral and neurodevelopmental abnormalities in offspring.

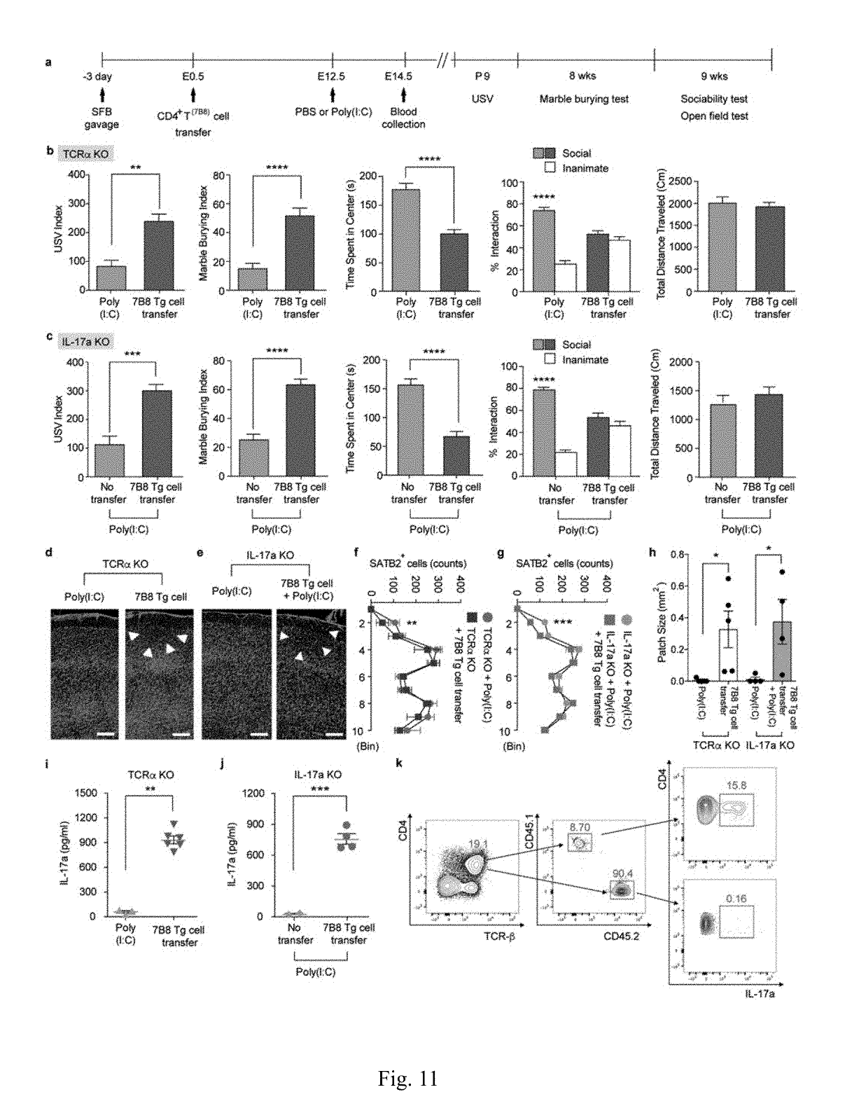

We next investigated whether commensal-antigen specific Th17 cells in pregnant mothers are sufficient to induce MIA-associated phenotypes in the offspring. Congenically marked naïve CD45.1⁺CD4⁺ T cells from mice expressing a transgenic T cell receptor (TCR) specific for a SFB-encoded antigen (7B8 Tg) were adoptively transferred into SFB-colonized CD45.2⁺ recipient mice lacking αβ T cells (TCRα KO) or deficient for IL-17a production (IL-17a KO) (FIG. 11a). The offspring derived from poly(I:C)-injected TCRα KO mothers crossed with B6 WT fathers failed to exhibit MIA-induced behavioral phenotypes. On the other hand, offspring from TCRα KO pregnant mothers that had received naïve 7B8 CD4⁺ T cells exhibited MIA-associated behavioral phenotypes even in the absence of exposure to poly(I:C)-induced inflammation (FIG. 11b). We subsequently tested if IL-17a produced by the SFB antigen-specific CD4⁺ T cells was sufficient to induce MIA phenotypes in offspring by transferring these cells into IL-17a KO females. Unlike the offspring from the poly(I:C)-treated IL-17a KO mothers that had been crossed with B6 WT fathers, offspring of poly(I:C)-injected IL-17a KO mothers that had received 7B8 CD4⁺ T cells displayed all four MIA-associated behavioral abnormalities (FIG. 11c). In addition, offspring from 7B8 CD4⁺ T cell recipient females exhibited the cortical phenotype (FIG. 11d-h). Induction of the MIA behavioral phenotypes was accompanied by an increase in IL-17a in the maternal plasma (FIG. 11i, j) and increased IL-17a production from SFB-specific donor CD45.1⁺ T cells, but not from CD45.2⁺ T cells of IL-17a KO recipient mice (FIG. 11k). Therefore, these results indicate that microbiota-specific gut Th17 cells present in pregnant mice are sufficient to produce abnormal behavioral and neurodevelopmental phenotypes in the offspring when accompanied by strong signaling for IL-17a production in the mother.

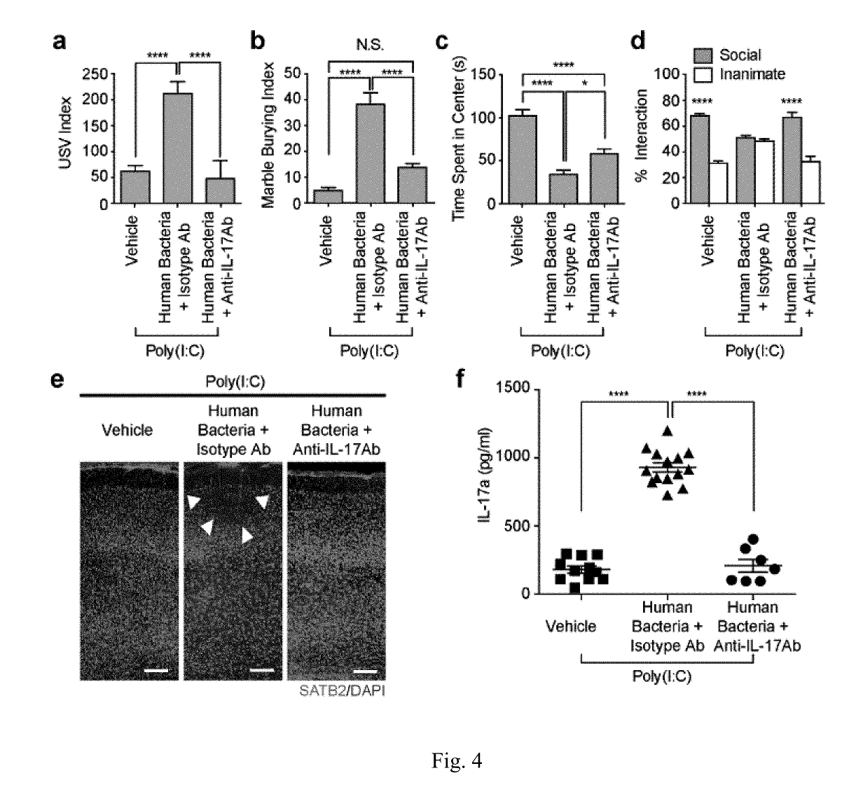

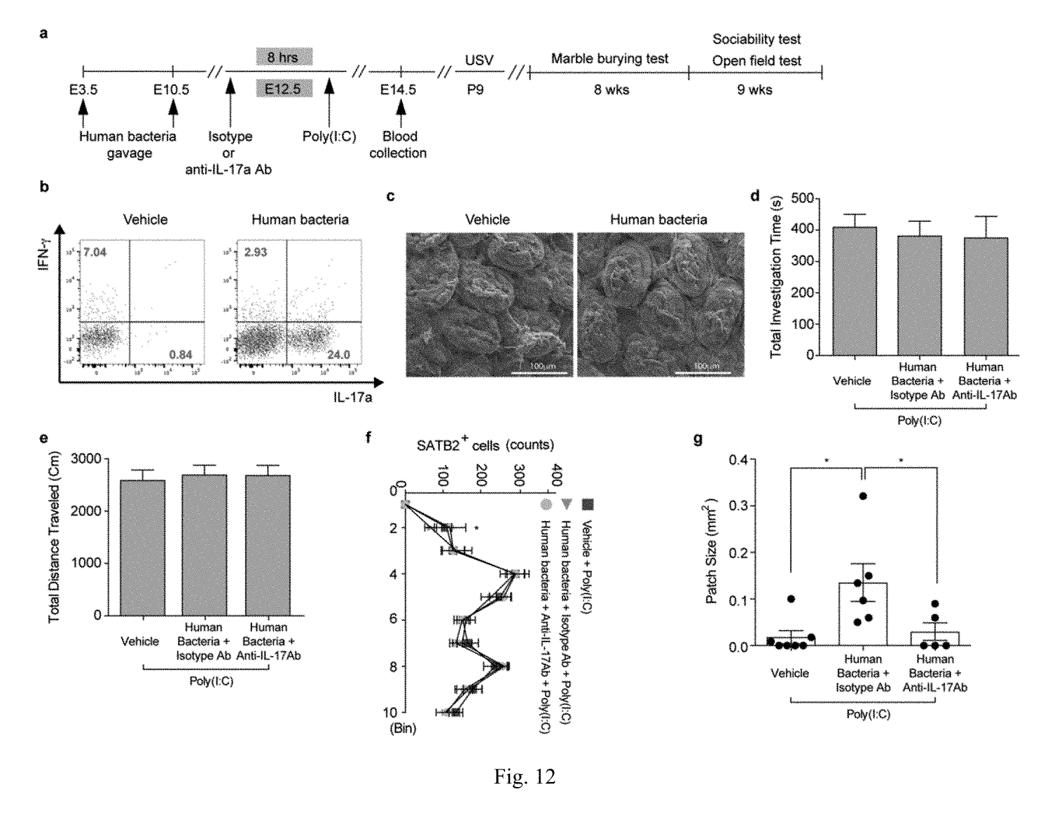

Further, we investigated if gut-residing bacteria isolated from humans could also promote MIA-associated phenotypes in mice. Administration of a mix of twenty different commensal bacteria isolated from human fecal samples was previously shown to induce Th17 cells in the large intestines of mice (Atarashi et al. Cell 163, 367-380, (2015)). We orally gavaged Jax mothers with a mix of these twenty human bacterial strains twice, on E3.5 and E10.5, followed by a poly(I:C) injection at E12.5 (FIG. 12a). Introduction of the twenty strains led to stable colonization of 2-10 commensal bacteria (Table 1) and to an increased percentage of Th17 cells in the colons of the recipient mice (FIG. 12b). Unlike in the SFB-colonized Jax mice, we could not detect SFB in the ilea of the recipient mice (FIG. 12c). Importantly, poly(I:C) injection of the human bacteria-gavaged Jax mice induced high levels of IL-17a in the maternal plasma and MIA-associated abnormal behavioral and neurodevelopmental phenotypes in the offspring (FIG. 4 and FIG. 12d-g). These MIA-associated phenotypes were not observed if the mothers were pre-treated with IL-17a blocking antibody (FIG. 4 and FIG. 12d-g).

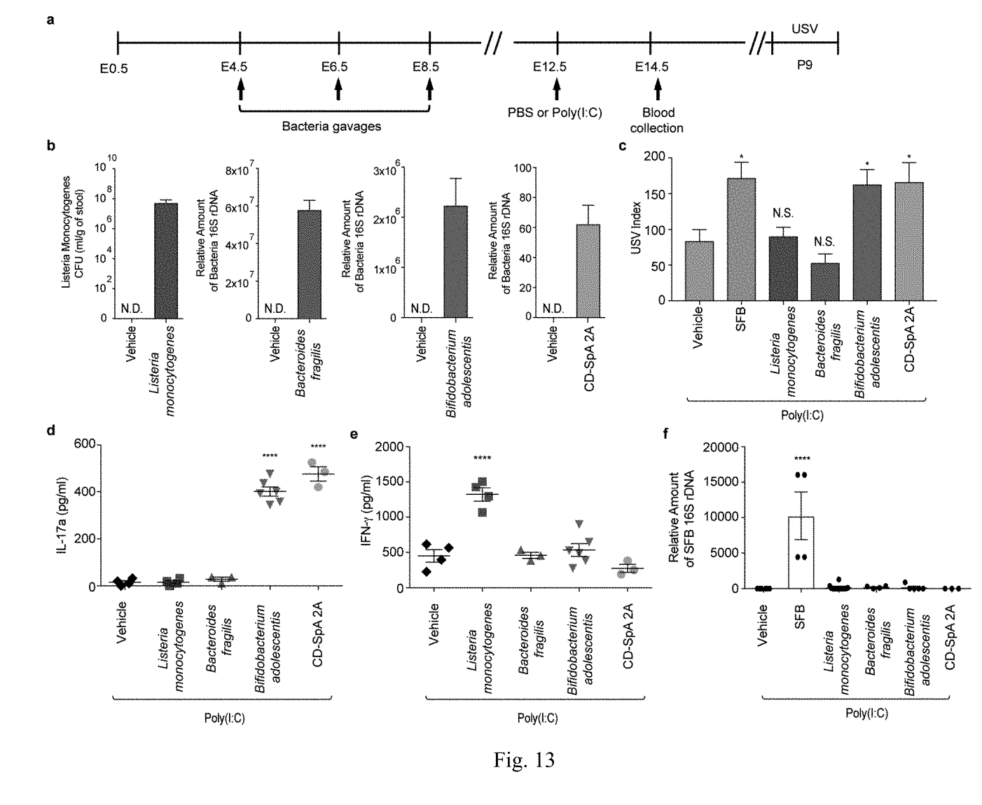

There have been several recent reports of individual human commensal bacteria that promote differentiation of intestinal Th17 cells. Unlike offspring from Jax mothers colonized with L. monocytogenes or B. fragilis, in which there is no induction of Th17 cells, offspring of mice colonized with Th17 cell-inducing B. adolescentis or an adherent E. coli isolate, CD-SpA 2A, emitted enhanced USV calls (FIG. 13a-c). The presence of MIA-associated behavioral phenotype in offspring correlated with increased IL-17a in the plasma of poly(I:C)-injected mothers (FIG. 13d). On the other hand, pregnant mice gavaged with L. monocytogenes had increased IFN-γ production (FIG. 13e). None of these human bacteria gavaged mothers were colonized with SFB (FIG. 13f). Thus, intestinal Th17 cell induction by individual human commensal bacteria contributes to the development of MIA-associated abnormality in mouse offspring.

The present disclosure describes the role of the microbiota in influencing the mother's risk of having offspring with neurodevelopmental disorders. Women with gut microbial communities that promote excessive Th17 cell differentiation may therefore be more likely to bear autistic children in the event of pathological inflammation during pregnancy. A better understanding of the role of the maternal microbiota and pregnancy-associated changes in gut-residing immune cells can provide opportunities to reduce the risk of inflammation-induced neurodevelopmental disorders.

Methods

Animals

All experiments were performed according to Guide for the Care and Use of Laboratory Animals and were approved by the National Institutes of Health and the Committee and Animal Care at University of Massachusetts Medical School. C57BL/6, tcra^{KO}, tlr3^{KO}, il17a^{gfp} and SFB-specific TCR Tg (7B8) mice were purchased from Taconic biosciences and Jackson Laboratory. To induce MIA phenotypes, SFB were introduced into mice purchased from Jackson laboratory. Il17a^{KO} mice were described in Nakae et al. Immunity 17, 375-387 (2002).

Maternal Immune Activation

Mice were mated overnight and females were checked daily for the presence of seminal plugs, noted as embryonic day 0.5. On E12.5, pregnant female mice were weighed and injected with a single dose (20 mg/kg; i.p.) of poly(I:C) (Sigma Aldrich) or PBS vehicle. Each dam was returned to its cage and left undisturbed until the birth of its litter. All pups remained with the mother until weaning on postnatal day 21-28 (P21-P28), at which time mice were group housed at a maximum of 5 per cage with same-sex littermates.

Co-Housing, SFB-Gavaged and Antibiotics-Gavaged Mice

For co-housing experiments, age-matched SFB-absent mice (from Jackson Laboratory) were co-housed with SFB-present mice (from Taconic Biosciences) in sterilized cages for two weeks at a ratio of 2:3, with unrestricted access to food and water. For SFB-gavaging experiments, four fecal pellets of SFB mono-colonized mice (provided by Dan Littman) were dissolved in 20 ml sterile PBS and filtered through a 100 μm cell strainer. 200 μl of fecal suspensions were gavaged via oral route to 4 week-old female Jackson mice. Control mice were gavaged with PBS. The SFB colonization was tested on day 7 following co-housing or SFB-gavaging. For ablation of intestinal bacteria, Taconic-derived female mice were orally gavaged with vancomycin hydrochloride (Fisher) (2.5 mg/kg) every two days, starting 7 days prior to breeding. Mouse fecal pellets were collected and stored at −80° C. before and after vancomycin treatments.

Human Commensal Bacteria-Gavaged Mice

Twenty human-associated Th17-inducing bacterial strains were isolated from fecal samples of a patient with ulcerative colitis. Fifteen strains (1A9, 1F8, 1D2, 1F7, 1D4, 2D9, 2E3, 2E1, 1D10, 1E3, 2H6, 2G4, 2G11, 1B11 and 1C2) were grown on Reinforced Clostridial Agar (Oxoid), two strains (1C12, 1E11) were on GAM Agar (Nissui), two strains (1D1, 2F7) were on Schaedler Agar (BD), and one strain (2H11) was on Tryptic Soy Agar (BD). Two days after plating, microbes were scraped from agar plates, suspended in 5 ml of 20% glycerol in PBS, and mixed with equal number of live bacteria (approximately final concentrations of 5×10⁸ CFU/ml of each strain). The mixture of twenty bacterial strains were stored at −80° C. until use. Pregnant Jax mice were inoculated twice by oral gavages at E3.5 and E10.5, with 200-300 μl of bacterial suspension. For the IL-17 cytokine blockade experiment, monoclonal IL-17a blocking antibody (clone 50104; R&D) or isotype control antibody (IgG2a, clone 54447; R&D) were administered 8 h before maternal immune activation via i.p. route (300 μg/animal). For colonization with B. Fragilis, B. Adolescentis and adherent E. coli CD-SpA 2A, pregnant Jax mice were inoculated three times by oral gavages at E4.5, E6.5 and E8.5 with 200 μl of bacteria suspension. (approximately final concentrations of 1×10⁹ CFU/ml of each strain). Bacterial stocks were prepared as previously described (Tan et al. Proc Natl Acad Sci USA 113, E8141-E8150, doi:10.1073/pnas.1617460113 (2016); Viladomiu et al. Sci Transl Med 9, doi:10.1126/scitranslmed.aa19655 (2017)).

Listeria-Gavaged Mice

Listeria monocytogenes was cultured in BHI broth media (Sigma, Cat no 35286 cfu). Pregnant Jax mice were inoculated three times by oral gavages at E4.5, E6.5 and E8.5 with 200 μl of bacteria suspension (approximately final concentrations of 2×10⁹ CFU/ml). Colonization levels were determined by collecting mouse fecal samples at E12.5, re-suspending them with PBS and subsequently plating on BHI agar.

Cross-Fostering

The day on which pups were born was considered P0. Pups were cross-fostered sometime between P0 and P1. Whole litters were removed from the original mothers. Pups were gently mixed with the bedding of the new cage. Pups were then introduced to the new cage with a foster mother. Pups from PBS- and poly(I:C)-treated Taconic mothers were cross-fostered to poly(I:C)- and PBS-treated Taconic mothers, respectively. Additionally, pups from Taconic- and Jax-derived mothers were cross-fostered to a Jax- and Taconic-derived dam, respectively.

Behavioral Assays

All behavioral testing were carried out according to the previously established behavioral schemes (Choi et al. Science 351, 933-939, (2016)) with minor modifications. Blinding was done for all the behavioral experiments except for the experiments with human bacteria.

Ultrasonic Vocalizations

On P9, both male and female offspring mice were removed from the nest and habituated to the testing room for 30 min. After the habituation period, mouse pups were placed in a clean 15 cm glass Pyrex high wall dish. Ultrasonic vocalizations (USVs) were detected for 3 min using an UltraSoundGateCM16/CMPA microphone (AviSoft) in the sound attenuation chamber under stable temperature and light control, and recorded with SAS Prolab software (AviSoft). USVs were measured between 33-125 kHz using Ultravox software (Noldus Information Technology, USA). Due to the unreliability of automated USV scoring, all pup USV calls were counted manually and plotted on the y-axis. Since both male and female pups of poly(I:C)-injected mothers emitted comparable levels of USVs (FIG. 5a), we did not separately analyze male versus female USV phenotypes. Both sexes were used for the experiments.

Three-Chamber Social Approach

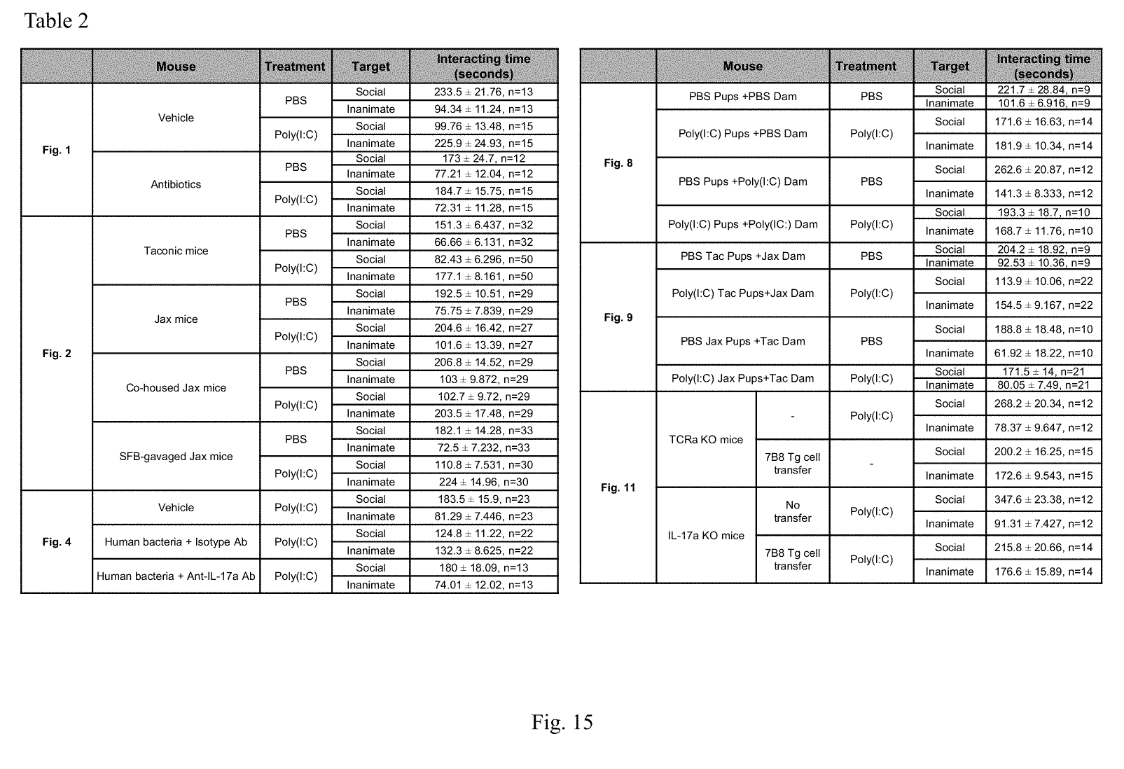

8-12-week-old male mice were tested for social behavior using the three-chamber social approach paradigm. Experimental mice were habituated for 1 h in separate clean holding cages and then introduced into a three-chamber arena with only empty object-containment cages (circular metallic cages, Stoelting Neuroscience) for a total 10-min acclimation phase in two 5 min sessions. The following day the mice were placed in the center chamber (without access to the left and right social test areas) and allowed to explore the center area for 5 min. After this exploration period, barriers to adjacent chambers were removed, allowing mice to explore the left and right arenas, which contained a social object (unfamiliar C57BL/6 male mouse) in one chamber and an inanimate object (black rubber stopper) in the other chamber. Experimental mice were given 10 min to explore both chambers and measured for approach behavior as interaction time (i.e. sniffing, approach) with targets in each chamber (within 2 cm). Sessions were video-recorded and object exploration time and total distance moved were analyzed using the Noldus tracking system. % interaction was calculated as the percentage of time spent investigating the social stimulus out of the total exploration time of both objects (Table 2, FIG. 15) and plotted on the y-axis. Arenas and contents were thoroughly cleaned between testing sessions. Multiple social targets from different home cages were used for testing to prevent potential odorant confounds from target home cages.

Marble Burying Test

Male mice were placed in a testing arena (arena size: 40×20 cm², bedding depth: 3 cm) containing 20 glass marbles, which were laid out in four rows of five marbles equidistant from one another. At the end of a 15 min exploration period, mice were gently removed from the testing cages and the number of marbles buried was recorded. A marble burying index was scored as 1 for marbles covered >50% by bedding, 0.5 for ˜50% covered, or 0 for anything less. Percentage of buried marbles is plotted on the y-axis.

Open Field Test

Mice underwent a 15-min exploration period in the testing arena (arena size: 50×50 cm²). Sessions were video-recorded and analyzed for time spent in the center (center size: 25×25 cm′) using EthoVision Noldus tracking system (Noldus, Netherlands). Time spent in the center of an open field is plotted on the y-axis.

Immunohistochemistry

Adult male mice were perfused and fixed with 4% paraformaldehyde in PBS for overnight at 4° C. The brains were removed and sectioned at 50 μm thickness with a Leica VT100S vibratome (Leica, USA). Slices were permeabilized with blocking solution containing 0.4% Triton X-100, 2% goat serum, and 1% BSA in PBS for 1 h at room temperature, and then incubated with anti-SATB2 (special AT-rich sequence-binding protein 2) (ab51502, Abcam) antibodies for overnight at 4° C. The following day, slices were incubated with fluorescently conjugated secondary antibodies (Invitrogen, USA) for 1 h at room temperature, and mounted in vectashield mounting medium with DAPI (Vector Laboratories). Images of stained brain slices were acquired using confocal microscope (LSM710. Carl Zeiss) with a 20× objective lens. The cortical malformation images were analyzed using Image J software. The images were cropped to have S1 cortical patches in the center.

Analysis of Cortical Patches

Cortical patches were identified as cortical regions devoid of SATB2 expression. The size of the cortical patches in the S1 was calculated using Zen software (Carl Zeiss). The cortical region was divided into 10 equal laminar blocks representing different depths of the cortex. SATB2 positive cells were quantified manually.

Scanning Electron Microscopy (SEM)

Terminal ileum tissues from mice (12-14 weeks old) were cut open and fixed with 2.5% glutaraldehyde in 0.1M cacodylate buffer (pH 7.4) for overnight and processed for standard SEM at EM center, University of Massachusetts Medical School. All samples were taken on a Hitachi S-4800 Type II Field Emission Scanning Electron Microscope.

16S rRNA Quantitative PCR Analysis

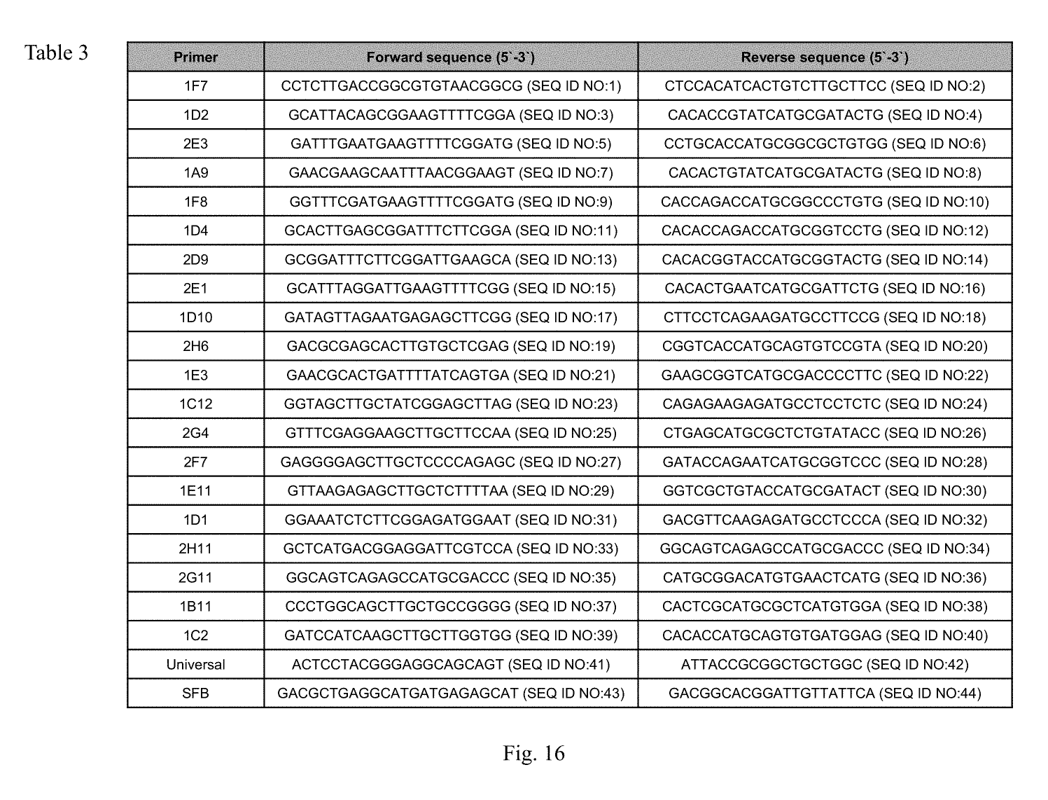

Bacterial genomic DNA was isolated from the fecal pellets of mice with phenol-chloroform extraction. qPCR was performed to quantify relative abundance of SFB, human commensal bacteria or total bacteria using group specific 16S rDNA primers (Table 3, FIG. 16). Undetected qPCR values from non-colonized samples were replaced with a Ct value of 40 for the purpose of comparison.

Lamina Propria Mononuclear Cell Preparation

For mononuclear cell isolations, both mesenteric fat tissues and Peyer's patches were carefully removed from intestinal tissues. Terminal ileal or colonic tissues were incubated in 5 mM EDTA in PBS containing 1 mM DTT at 37° C. on a shaker (200 rpm) for 20 min. Tissues were washed one more time. Tissues were then further digested for 30 min at 37° C. in RPMI containing 10% fetal bovine serum, 1.0 mg/ml Collagenase D (Roche) and 100 μg/ml DNase I (Sigma). Digested tissues were then filtered using a 100 μm cell strainer and incubated for additional 10 min at 37° C. Mononuclear cells were isolated from an interphase of percoll gradients (40:80 gradient).

Flow Cytometry

Mononuclear cells were incubated with or without 50 ng/ml phorbol myristate acetate (PMA) (Sigma) and 500 ng/ml ionomycin (Sigma) in the presence of GolgiStop (BD) in complete T cell media at 37° C. for 5 h. Intracellular cytokine staining was performed according to the manufacturer's protocol. Cells were stained with Pacific Blue-conjugated anti-CD4 (RM-5), PerCP-Cy5.5-conjugated anti-CD8a (53-6.7), APC-Cy7-conjugated anti-TCRβ (H57-597), FITC-conjugated anti-CD62L (MEL-14), APC-conjugated anti-CD44 (IM7), PE-conjugated-CD25 (PC61.5), PerCP-Cy5.5-conjugated-CD19 (eBio1D3), APC-conjugated anti-CD45.1 (A20), FITC-conjugated anti-CD45.2 (104), Pacific Blue-conjugated anti-CD11c (N418), FITC conjugated-anti-CD11b (M1/70), PerCP-Cy5.5-conjugated-anti-CD103 (2E7) (eBioscience), Biotin-conjugated Vβ14 (14-2) (BD phamigen) and PE-Cy7-conjugated-Streptavidin (Thermo Fisher Scientific). Cells were further stained intracellularly with APC-conjugated anti-RORγ (B2D) (eBioscience) and PE-Cy7-conjugated anti-IL-17a (eBio17B7) (eBioscience) using Foxp3 staining/permeabilization buffer (eBioscience). Flow cytometric analysis was performed on an LSRII (BD Biosciences). All data were re-analyzed using FlowJo (Tree Star).

Adoptive Transfer

Spleen and lymph nodes from 7B8 Tg mice were collected and disassociated. Red blood cells were lysed using ACK lysis buffer (Lonza). Naïve CD4⁺ T cells (CD62L^{hi} CD44^{lo} TCRvβ14⁺CD4⁺CD19⁻) from CD45.1⁺7B8 Tg mice were sorted on a BD FACS Aria II. Sorted 5×10⁴ cells were transferred into congenic CD45.2 recipient mice by tail vein injection.

Cell Sorting

Mononuclear cells were isolated at E14.5 from small intestines of poly(I:C)-treated pregnant Il-17a^{gfp} or B6 mice. GFP⁺ and GFP⁻ T cells, gated on CD8⁻CD19⁻TCRβ⁺CD4⁺, were sorted with a FACSAria (BD biosciences). DCs were stained with antibodies and sorted based on their surface expression of CD103 and CD11b (gated on CD4⁻CD8⁻CD19⁻MHCII⁺CD11c⁺.

Ex Vivo Mononuclear Cell Culture

Mononuclear cells isolated from ilea of either PBS- or poly(I:C)-treated mice on E14.5 were cultured in vitro with poly(I:C) (2.5 μg/ml). CD4⁺ or CD11c⁺ cells were positively selected using microbeads (Miltenyi). For co-culture assay, CD4⁺ cells (1.5-3.5×10⁴ cells/mL) were cultured with CD11c⁺ cells (7.5-16×10⁴ cells/ml) at 1:5 ratio in each well. CD4⁺ and CD11c⁺ cells were incubated for 24-48 h with IgG antibody (20 ng/ml) or with anti-IL-1(3 antibody (20 ng/ml), anti-IL-6 antibody (20 ng/ml) and anti-IL-23p19 antibody (20 ng/ml) (R&D System) with or without poly(I:C) stimulation (2.5 μg/ml) (Sigma). CD4⁺ cells were cultured with recombinant IL-1(3 (long/ml), IL-6 (5 ng/ml) and IL-23 (5 ng/ml) (R&D System). All cells were cultured in T cell media: RPMI 1640 (Invitrogen) supplemented with 10% (v/v) heat-inactivated FBS (Hyclone) and 50 U penicillin-streptomycin (Invitrogen). Cell culture supernatant was used for ELISA analyses.

ELISA

IL-17a, TNF-α, IFN-β, IL-1β and IL-23 levels were measured according to the manufacturer's protocol (BioLegend). IL-6 and IFN-γ levels were measured according to the manufacturer's protocol (eBioscience).

Statistics

Statistical analyses were performed using GraphPad Prism. ANOVAs were followed by Tukey or Sidak tests. All data are represented as mean+/−SEM. Sample sizes were determined based on similarly conducted studies (Choi et al. Science 351, 933-939, doi:10.1126/science.aad0314 (2016)). When conducting behavioral assays, cages were pseudo-randomly assigned for tests. Detailed statistical analyses for behavioral assays are listed below.

While the present invention has been described through various specific embodiments, routine modification to these embodiments will be apparent to those skilled in the art, which modifications are intended to be included within the scope of this disclosure.

What is claimed is:

1. A method of reducing the risk of a female subject producing an offspring having a neurodevelopmental disorder comprising:
a) in a sample comprising gut bacteria obtained from the female subject, testing for the presence of bacteria that promote Th17 cell biogenesis; and
b) if the presence of such bacteria is detected, administering to the female subject, a therapy that inhibits the bacteria, reduces the Th17 cell biogenesis promoting activity of said bacteria, and/or reduces the activity of Th17 cells in the gut.

2. The method of claim 1, further comprising the step of testing the effectiveness in vitro of a therapy that inhibits Th17 cell biogenesis promoting bacteria or reduces Th17 cell biogenesis promoting activity of said bacteria, prior to administering the identified agents to the female subject.

3. The method of claim 1, wherein the therapy that inhibits the bacteria comprises administering a composition comprising an antibiotic.

4. The method of claim 3, wherein the antibiotic is a broad spectrum antibiotic.

5. The method of claim 4, wherein the broad-spectrum antibiotic is vancomycin.

6. The method of claim 3, wherein the antibiotic is ampicillin, metronidazole, amoxicillin, levofloxacin, gatifloxacillin, streptomycin, tetracycline, chloramphenicol, or combinations thereof.

7. The method of claim 1, wherein the sample is tested for the presence of one or more strains of bacteria from the species Clostridium, Coprobacillus, Eubacterium, Erysipelotrichaceae, Firmicutes, Subdoligranulum, Butyrate producing bacterium, Bifidobacterium, Ruminococcus, and/or Bacteroides, or combinations thereof.

8. The method of claim 1, wherein the sample is tested for the presence of one or more strains of bacteria listed in Table 1.

9. The method of claim 1, wherein the sample is tested for the presence of one or more strains of B. adolescentis, or an E. coli isolate, CD-SpA 2A.

10. The method of claim 1, wherein the sample is a fecal sample.

11. The method of claim 10, further comprising the step of obtaining a fecal sample from the female subject.

12. The method of claim 1, wherein the sample is a sample of the content or tissue from the gut.

13. The method of claim 1, wherein the testing is carried out prior to pregnancy or during pregnancy.

14. The method of claim 1, further comprising the step of obtaining a sample comprising gut bacteria from the female subject.

15. The method of claim 1, wherein if the presence of bacteria that promote Th17 cell biogenesis is detected in a fecal sample from a pregnant female subject, administering to the offspring a therapy comprising a pharmacological agent or implementing optogenetic or chemogenetic tools that corrects dysregulated neuronal excitation/inhibition (E/I) ratios in the cortex of the offspring, wherein the therapy is administered in utero or after birth.

16. The method of claim 15, wherein the pharmacological agent is a GABAergic receptor agonist.

17. The method of claim 16, wherein the GABAergic receptor agonist is synthetic GABA, muscimol, a barbiturate, or a benzodiazapine.

18. The method of claim 15, wherein the optogenetic tools are channelrhodopsin, halorhodopsin, or an OptoXR.

19. A method of identifying/implementing an individualized treatment plan for a female subject to reduce the risk of the female subject producing an offspring with a neurodevelopmental disorder comprising:
a) identifying in the gut of the female subject, the presence of one or more types of bacteria that promote Th17 cell biogenesis, wherein the presence of one of more of the said bacteria indicates an increased likelihood that the female subject will produce an offspring with a neurodevelopmental disorder; and
b) if the presence of said bacteria is determined, then screening said bacteria for:
i) effective anti-bacterial agents, and/or
ii) effective Th17 cell suppressing agents,
wherein the identified effective anti-bacterial agents inhibit the growth of the one or more bacteria that promote Th17 cell biogenesis, and the effective Th17 cell suppressing agents inhibit the number or activity of the gut Th17 cells.

20. The method of claim 19, further comprising administering the identified effective anti-bacterial agents that inhibit the growth of the one or more bacteria that promote Th17 cell biogenesis, or the effective Th17 cell suppressing agents that inhibit the number or activity of the gut Th17 cells to the female subject.
